(19) United States
(12) Patent Application Publication
(10) Pub. No.: US 2003/0216795 A1
(43) Pub. Date: Nov. 20, 2003

(54) Apparatus and method for high energy photodynamic therapy of acne vulgaris, seborrhea and other skin disorders

(76) Inventors: Yoram Harth, Haifa (IL); Avner Korman, Herzlia (IL)

Correspondence Address: WATOV & KIPNES, P.C., P.O. Box 247, Princeton Junction, NJ, 08550, US

(21) Appl. No.: 10/366,452

(22) Filed: Feb. 13, 2003

Related U.S. Application Data
(63) Continuation-in-part of application No. 10/007,702, filed on Dec. 10, 2001.
Application No. 10/007,702 is a continuation-in-part of application No. 09/756,130, filed on Jan. 9, 2001.
Application No. 09/756,130 is a continuation-in-part of application No. PCT/IL99/00374, filed on Jul. 7, 1999.

(51) Int. Cl. A61N 1/00

(52) U.S. Cl. 607/88

(57) ABSTRACT

An apparatus, a device, a system and a method for the phototherapy of different skin disorders including acne vulgaris, seborrhea and inflammation. The invention includes a multiple session treatment method by a high irradiance violet/blue CW time gated, or pulsed, light source with a spectral emission in the range of 400-450 nanometer and possible additional spectral bands in the green and red part of the spectrum. The apparatus includes at least one narrow spectral band light source with spectral emittance concentrated in the violet/blue spectral band. The invention can reduce the level of extra cellular pro inflammatory cytokines related to inflammation as well as to significantly reduce the acne bacteria population by a photodynamic effect. A system for the treatment of acne lesions combining the apparatus for the eradication of P. acne bacteria and the acne affected area inflammation and a device for the elimination of the red spots around the acne lesions using intense pulsed light in the spectral band 500-1000 nm.

### RELATED APPLICATIONS

This application is a continuation in part application of U.S. application Ser. No. 10/007,702, filed Dec. 10, 2001, which is a continuation in part application of U.S. application Ser. No. 09/756,130, filed Jan. 9, 2001, which is a continuation in part application of PCT Application No: PCT/IL99/00374, filed Jul. 7, 1999.

### FIELD AND BACKGROUND OF THE INVENTION

The present invention relates to an apparatus and method for the photodynamic treatment of acne vulgaris and seborrhea and conditions and symptoms associated therewith as well as other related skin disorders hereinafter referred to at times as “acne”, more particularly, to a violet/blue light radiating system that illuminates a high flux narrow bandwidth beam on the treated skin area. The method relates to the photodynamic skin treatment using the narrow band violet/blue light apparatus of the invention. The present invention also relates to the phototherapy of inflammation and particularly to the phototherapy of inflammation using narrow band high intensity light source in the spectral range of 400-450 nm.

The invention also relates to the comprehensive treatment of acne skin condition, and more particularly to the combined treatment of acne inflammatory lesions with the reduction of acne lesion localized reddish and irritated skin condition, The post acute acne stage of the typical lesion area is characterized by localized skin redness. The treatment of post acute inflammation treatment phase is naturally consecutive to the preliminary stage of anti bacterial treatment of the inflammatory acne lesion itself.

The enlargement and obstruction of sebaceous glands cause acne vulgaris. Due to the accumulation of sebum in the glands, bacteria, mainly propionibacterium acnes (p. acnes), proliferate in the glands. These bacteria cause inflammation and later the formation of pustular lesions and acne cysts, which heal by scarring.

It is known that p. acnes produce porphyrins. It is also known that visible light in the violet/blue (405-410 nanometer range), or less efficiently, red (630-670 nanometer range) are able to induce a photodynamic effect in which the porphyrins in the enlarged sebaceous glands react with oxygen to form peroxides. These peroxides are short-lived toxic compounds that are able to eliminate, or considerably diminish, the number of bacteria in the glands.

Photodynamic therapy (PDT) is based on the optimal interaction of four elements; light, photosensitizer, oxygen and skin penetration. Prior patents and publications related to acne phototherapy dealt only with the first two elements of PDT, i.e., and light exposure and sebaceous gland porphyrins. Studies have shown that the photodestruction of p. acnes is increased exponentially in an oxygen rich environment.

Various attempts have been made to treat acne with light; Mendes et al. (U.S. Pat. No. 5,549,660) described a method for the light therapy of acne using low intensity red light. Their apparatus was meant to treat acne through it effect on macrophages in the skin. Its low light intensity is not sufficient for an efficient photodynamic destruction of p. acnes in the deeper layers of the skin. High intensity visible light phototherapy for acne was described by Meffert et al., (Dermatol-Monatsschr. 1990; 176(10): 597-603) but they used a light source emitting not only visible light but also UVA comprising up to 15-20% of the total irradiation dose. Sigurdsson et al (Dermatology 1997; 94:256-260), used Philips HPM-10 400W combined with an UVILEX 390-filter (Desag. Germany) that filters most but not all ultraviolet A (UVA) harmful rays. The spectrum of their lamp peaked at 420 nanometer and had 2 other small peak of emission at 405 and 435 nanometer. Their apparatus emitted at 40 cm; 0.5 J/cm² of UVA, 20 J/cm²/of violet/blue and 5 J/cm² of green. None of the prior art devices aimed at the photo treatment of acne free of harmful UV radiation are able to essentially eliminate the acne lesions within a short period of four weeks in the vast majority of patients. All photo based treatments necessitates at least 3-4 month of treatment procedures resulting in increasing risks of scars. Low power photo treatment of inflammations is practiced for over 2 decades. The spectral range used for these treatments is in the red (630 nm) or the Infrared range of the spectrum. Other lamps or light sources used for the treatment of inflammations emit blue light but in conjunction with ultraviolet light. The use and intensity of these sources is limited by the erythemogenic, photoaging and photo carcinogenic effects of UV light.

High intensity, UV free, violet/blue light sources are used for the therapy of Acne, whereby p. acnes bacteria are destroyed through a photodynamic process (previous application of this CIP—International patent publication PCT No. WO 00/02491).

The selective treatment of vascular lesions with specific lasers which emit energy in defined narrow spectral bands that are selectively absorbed by the blood, is well known in the prior art. Light absorption is dominated by the hemoglobins and melanin. Tissue water plays an insignificant role in the spectral region of interest from 650-1000 nm. Melanin has a monotonically decreasing absorption profile from the ultraviolet region to the near infra-red region, where absorption becomes virtually negligible around 1000 nm. The hemoglobins also tend to have an absorption characteristic which decreases towards the infra-red, though it has localized absorption peaks corresponding to molecular and atomic transitions. Oxyhemoglobin has absorption peaks around 430, 540 and 577 nm, while deoxygenated hemoglobin has absorption peaks around 450 and 560 nm. The absorption characteristics of whole blood is considered to be partially oxygenated, at the 80% level giving a preferred absorption band in the range 450-600 nm. Other blood borne chromophores include bilirubin, which has a broad absorption band around 450 nm, and beta-carotene, with a broad absorption band in the region 450-500 nm. An optical window, with minimal absorption and scattering, exists between 600-1000 nm, where depth of penetration extends to several mm.

In U.S. Pat. No. 4,971,486, Raven et al., blood vessels are eliminated using infrared radiation. U.S. Pat. No. 5,630,811, Miller et al. describes the effect of photocoagulation using a laser. U.S. Pat. No. 5,707,403 Grove et al. is also covering aspects of coagulation of blood vessels by lasers, including laser diodes.

U.S. Pat. No. 4,829,262, Furumote describes a tunable dye laser implemented for selective photothermolysis.

The selective treatment of vascular lesions with the aid of non coherent broad spectrum Intense Pulsed Light (IPL) systems, is also known in the prior art like; U.S. Pat. Nos. 5,626,631, 6,280,438 and 6,174,325 all by Eckhouse et al. each of which is incorporated herein by reference. The system in U.S. Pat. No. 6,280,438 includes pulsed light sources such as flash lamps for providing electromagnetic treatment of the skin, including hair removal. It also targets a wide range of vascular lesions, including deep vascular lesions such as leg veins in 1-3 mm depth, Telangectasia in 0.5 mm depth, as well as large surface port wine stains. Since typical affected areas are comparatively large in size, the required treatment exposed surface per pulse is large; often over 2-3 cm square. The faster the system can cover and treat large areas the more economical it is. In practice this area coverage parameter defines the treatment acceptance and the greater the chances are that the system is successfully marketed.

Another related treatment modality has evolved which is generally called Photorejuvenation, such a process is described in U.S. Pat. No. 6,120,497, Mcmillan et al., incorporated herein by reference covering a method for treating wrinkles in a skin involving the use of a beam of pulsed, scanned or gated continuous wave laser or incoherent radiation. A system called Quantum IPL (marketed by Lumenis Corp, USA) is capable, by radiating high energy short duration visible light, of erasing superficial wrinkles, reducing the size of skin pores and reducing the redness of skin sites. The system is based on a broad spectral band IPL which heats collagen and heats blood vessels. The effect of heating of collagen stimulates collagen contraction and remodeling, resulting in wrinkle removal. The IPL light is also absorbed by pigment cells in the epidermis, thereby gradually reducing pigmentation. The IPL light is also absorbed in skin pores, thereby gradually stimulating collagen and contracting the pores. The present art IPL light is also slightly absorbed by the skin red spots, which are rich with vascularization, thereby coagulating the blood vessels without damaging the skin tissue cells in the immediate neighborhood. The number of treatments necessary to attain noticeable results with these IPL devices typically range between 10 to 20 J/cm² per pulse.

Photorejuvenation is also described in the publications of the CoolTouch® and Thermescent Skin Treatments™, by ICN Pharmaceuticals Inc, from Costa Mesa, Calif. USA, which offer non-invasive process to stimulate the production of new collagen to reduce fine lines and wrinkles and combat the effects of aging. A similar approach is described in U.S. Pat. No. 6,387,089 by Eckhouse et al. incorporated herein by reference. In all described devices and methods, large skin surfaces are treated to obtain visible results. The main reason for the necessity to treat large areas with Photorejuvenation devices is the marginal noticeable results, while considering specifically the results in each spot. This low profile results are in contrast to the acceptance of high improvement in the general treated skin condition, when those minimal results are accumulated over the entire face. Also treatment time should be relatively fast due to operational and economical demands.

All present art IPL Photorejuvenation treatment systems have a treatment exposure spot size larger than ˜2 cm², and have typical radiation treatment energy per pulse in the range of 10-40 J/cm². Most of its energy content as a percentage of the emitted pulse energy is in the visible and the Infrared spectral content, to enable both vascular and collagen heating. Production costs of these devices are therefore high due to necessity to assure high output treatment energy per pulse and a large enough treatment spot size.

The prior art IPL systems suffer from an additional disadvantage. The energy density per pulse exposure (J/cm²) of the typical commercial IPL systems used for Photorejuvenation (15-40 J/cm²) are also high enough to cause the removal of exposed hairs. As a result the prior art systems cannot be used on the faces of males for treatment applications concerning vascular and collagen heating. As an example, prior art IPL systems cannot be used to erase acne lesions by eradication of the P. acne bacteria as well as the reduction of post acne skin redness from acne lesions on male faces, since such a treatment will also permanently remove the male facial hairs.

A dermatological condition which requires a fast and effective lesion destruction and local redness elimination treatment, is inflammatory acne. The advantages of the enhanced blue/violet light based acne treatment devices have been demonstrated to clear acne inflammatory stage lesions within a typical treatment session of 6-10 treatments within a period of one month (Clearlight™ by Curelight ltd., distributed by Lumenis Corp, USA. As described in Applicants' co-pending U.S. application Ser. No. 09/756,130, filed Jan. 9, 2001). The advantage of the fast eradication of P. acne bacteria in acne lesions is the reduction of post acne scar risk. However, a disadvantage of the present art blue/violet light based device, is the lack of clearance of strong skin redness localized areas which follows post acne acute inflammatory lesion condition. The skin redness is generally created by enlarged blood micro capillaries which were produced around the acne lesion localized inflammation, created during the acute acne condition.

It would be highly advantageous to have a device which would combine the eradication of the P. acne bacteria together with erasing the post acne lesion localized skin redness, without affecting in any way the skin around the post acne typical lesion, which is typically of 4 mm diameter in size.

Prior art IPL systems utilize high energy Xenon flash lamp technology, or high intensity laser light sources in the yellow to NIR range of the spectrum (500-700 nm). There are no violet/blue IPL based systems to take advantage of the high efficiency photodynamic effect in which the P. acne induced porphyrins in the sebaceous glands react with violet/blue light energy and destroy the P. acne bacteria, by the formation of peroxides. There is therefore a need to have an acne treatment method and system, wherein an intense pulsed light device will be integrated as a part of an acne phototherapy system and a related treatment method will provide the capability to effectively and economically treat acne lesions in all the skin condition development stages, including the post inflammatory small, locally affected red skin areas. These red skin areas should preferably be treated without affecting the surrounding tissues and without causing damages such as removal of hair or creation an unwanted hyper or hypo-pigmentation.

### SUMMARY OF THE INVENTION

According to the present invention there is provided an apparatus and a method for acne phototherapy, achieved by the use of a specially designed apparatus having a narrow spectral band of violet/blue light emission radiation in the range 400-470 nm, preferably in the range 400-450 nm with the radiation characterized by a high energy threshold level, above pre-determined radiation flux the light source of the apparatus can be a gated CW light source or a narrow high peak power pulsed low repetition rate light source. In another embodiment said apparatus having at least one additional spectral line. In another embodiment the illumination of the light source flux, within the entire UV spectrum, is less than a predetermined UV safety energy threshold level.

There is also provided a focused spot IPL device that can enable the localized selective exposure treatment of very small red spots with energy spectrum mainly in the spectral range of 500-700 nm for the reduction of the lesion inflammation redness and which is operated in conjunction with the treatment of acne in the spectral band of 400-450 nm and is an integrated part of an acne treatment light based system according to this invention.

In another embodiment the illumination flux of the light source in the UV spectrum is less than 0.1 microwatt/cm² in the spectral range of 200-400 nm.

In another embodiment the treatment with the invention apparatus can be combined with a pre-treatment application on the treated skin area of an oxygen transporting compound, based on the use of one or more of the materials from the group of compounds consisting of perfluorocarbons, oxidative substances, keratolytic substances and external photosensitizer, such as Methylene blue 0.1-5%.

The present invention apparatus utilizes in another embodiment the least one spectral band for the treatment of inflammation. This spectral band is a high intensity, UV free, narrowband violet/blue light energy in the spectral band range of 400-450 nm.

The invention apparatus adaptation for the treatment of inflammation is based on the research discovery that the violet-blue light at the same spectral bandwidth as the light applicable for the treatment of acne is also capable of reducing proinflammatory cytokines produced by T cells, thereby diminishing local inflammation.

It was further found that by illuminating inflamed tissues, including the case of inflamed acne lesions, with at least 5 J/cm² of light within the bandwidth of 400-450 nm, the inflammation is significantly diminished. That inflammation reduction discovery paves the way to the efficient, safe and fast therapy of large areas as well as lesional targeted areas of inflamed tissues without the risks of UV irradiation.

There is thus provided, in accordance with an embodiment of the present invention, apparatus for treatment of a skin disorder. The apparatus includes at least one light source with spectral emittance concentrated in at least one specific narrow spectral band, wherein one spectral band is in the range of 400 to 450 nm, an optical system for collecting and shaping light emitted from the at least one light source and an electronic unit to control parameters associated with the spectral emittance from the at least one light source.

Furthermore, in accordance with an embodiment of the present invention, the parameters include at least one of a group including duration, power density, energy per pulse and emitted spectral bands of the light source emittance.

Furthermore, in accordance with an embodiment of the present invention, the apparatus further includes a mechanical fixture for holding the light source at an adjustable distance and direction related to a treatment area.

Furthermore, in accordance with an embodiment of the present invention, the illumination energy of the light source flux, is higher than a predetermined threshold level. The threshold level is a level required for biological destruction of acne and seborrhea causing factors. Specifically this treatment energy threshold level enables the destruction of the P. acne bacteria population to a much lower level, so as to get a significantly reduced post treatment bacteria population size.

The invention apparatus illumination energy flux level triggers a photodynamic treatment process that creates the condition in which the acne bacteria destruction rate surpasses the natural bacteria population average multiplication and growth rate, thus ensuring that the balance of the bacteria population will be significantly further reduced from one treatment to another, until total bacteria population reduction ratio will provide treatment results wherein post-treatment bacteria population will be at least four (4) orders of magnitudes lower than the pre-treatment bacteria population size.

Furthermore, in accordance with an embodiment of the present invention the invention apparatus for the treatment of acne is capable of reducing over 60% of the original number of acne lesions in average within four weeks, without radiating any UV illumination on the treated skin area and without causing any damage to this skin.

Furthermore, in accordance with an embodiment of the present invention, the illumination energy threshold level of the illumination light source is at least 40 mw/cm². Furthermore, in accordance with an embodiment of the present invention, the illuminated area on a patient body includes an illumination area large enough to illuminate an infected typical size skin area from a fixed position of the light source related to the skin area. In accordance with an embodiment of the present invention, the illuminated area is at least 400 cm².

Furthermore, in accordance with yet another embodiment of the present invention, while using a high peak power pulsed light as the light source the illuminated area on a patient body is defined by the area of the beam created by implementing a direct contact between the illumination source and the treated skin.

Additionally, in accordance with an embodiment of the present invention, the apparatus further includes an illumination head having at least two converging collimated beams from at least two directions, each of the beams generated by a separate light source positioned at a distance from the other at least one light source.

Additionally, in accordance with an embodiment of the present invention, the apparatus further includes a computer controlled imaging unit for imaging an illuminated treated area and for monitoring by counting lesions on the treated area, using computerized counting techniques.

Additionally, in accordance with an embodiment of the present invention, the apparatus further includes a computer controlled display unit for displaying the imaged illumination treated area, wherein counting is carried out by an operator marking lesions on the display of the illumination treated imaged surface area. Alternatively, the computer lesions counting by image processing techniques to detect and count each lesion in the illumination treated imaged surface area. The score of the computer lesion counting is recorded in a computer memory to enable monitoring the lesion healing process through a series of consecutive treatments.

Furthermore, in accordance with an embodiment of the present invention, the computer controlled imaging unit display image includes at least one of a group includes a graph of the number of counted lesions versus accumulated treatment time and a table consisting of number of counted lesions in each treatment session.

Furthermore, in accordance with an embodiment of the present invention, the apparatus further includes at least one optical element of a group includes a liquid filled light guide, a solid transparent light guide, a fiber bundle light guide and an array of lenses and mirrors for collecting and conducting the light source radiation and illuminating the skin treated area at an adjustable distance, energy density and direction.

Furthermore, in accordance with an embodiment of the present invention, the light source is a Gallium, Mercury and at least one gaseous halide discharge lamp with peak emission in the 400-450 spectral band. Alternatively, the light source is an Excimer lamp with peak emission in the spectral range of 400-430 nm.

Alternatively, the light source is composed of one or more of a group of light sources including a metal halide gas discharge lamp, a xenon gas discharge lamp, an excimer lamp, an Ion Krypton gas laser with a spectral emission in the range 400 to 450 nm, a diode and a diode matrix. The diode or diodes are selected from the group consisting of violet/blue laser diodes, and light emitting diodes (LED) with narrow spectral band emission in the spectral range of 405-440 nm, or 520-570 nm, or 625-670 nm, or any combination thereof.

Furthermore, in accordance with an embodiment of the present invention, the light is collected and projected by at least one reflector, wherein the reflector is selected from the group includes of an elliptical cross-section cylindrical reflector, parabolic cross-section cylindrical reflector, and an asymmetric aspheric reflector.

Alternatively, the light is collected and further collimated by a set of two orthogonal cylindrical lenses.

Furthermore, in accordance with an embodiment of the present invention, the light of the at least one light source is collected by an elliptical cross-section reflector having a first focal point and a second focal point. The light source is disposed at the first focal point and has disposed at the second focal point a slit shape aperture of a slit to circular beam shaping and conducting light guide.

In another embodiment of the invention, the apparatus is used for the reduction of inflammation, wherein the output power spectral band is further broadened to the 400-450 nm spectral range.

In yet another embodiment of the invention apparatus, the light source is capable of treating any skin disorder related to inflammation from the group including; soft tissue inflammation, muscular inflammation, post traumatic soft tissue and muscular pains, arthralgia, skin ulcers such as diabetic ulcers and stasis ulcers, contact dermatitis, atopic dermatitis and systemic and localized scleroderma.

In yet another embodiment of the invention, apparatus the output radiation spectrum is UV free.

In yet another embodiment of the invention, apparatus at least one light source is selected from the group that includes a Gallium, Mercury and halides gas mixture discharge lamp and a Xenon gas discharge lamp with peak emission in the 400-450 spectral band.

In another embodiment of the invention the use of the apparatus for the treatment of inflammation; and (a) the light radiation source having spectral characteristics that contain at least radiation energy in the narrow band of violet/blue (400-450 nm); and (b) the light source having energy output flux over a predetermined threshold level.

In another embodiment of the invention the use of the apparatus for the treatment of inflammation wherein the; output flux predetermined threshold level is 10 mW/cm².

In another embodiment of the invention the use of the apparatus for the treatment of inflammation wherein the; the step of illuminating is accomplished by projecting on said skin area with an illumination power in the range of at least 10 mW/cm² of violet/blue light radiation.

In another embodiment of the invention the use of the apparatus for the treatment of inflammation wherein the; the treated area is larger than 2 cm×2 cm. In another embodiment of the invention the use of the apparatus for the treatment of inflammation wherein the; the light radiation source is capable of reducing level of extra cellular pro-inflammatory cytokines.

In another embodiment of the invention the use of the apparatus for the treatment of inflammation wherein the; cytokines are reduced by an amount of at least 70% of the reduction achieved by UVB at the same energy radiation conditions.

In another embodiment of the invention the use of the apparatus for the treatment of inflammation wherein the light radiation source is capable of treating skin ulcers.

In another embodiment of the invention the use of the apparatus for the treatment of inflammation wherein the treatment is performed in conjunction with mechanical debridment of tissue i.e. mechanical, chemical, by high pressure later jet or ultrasonic waves.

In another embodiment of the invention the use of the apparatus for the treatment of inflammation wherein the treatment requires a predetermined time period of 1-5 weekly exposures to violet/blue light for typically 1-10 weeks, with a minimum 24 hour's time gap between exposures.

In another embodiment of the invention the use of the apparatus for the treatment of inflammation wherein the step of illuminating is accomplished by projecting on said skin area with an illumination power in the range of at least 10 mW/cm² of violet/blue light radiation.

The present invention device is so designed and has the specific capability to treat and eliminate micro size veins by coagulating the sub millimeter size blood vessels without damaging the skin tissue cells in the immediate distance surrounding tissue. These micro veins of less than 0.1 mm in diameter are best coagulated at a short pulse radiation in the spectral band 520-700 nm. The device is targeted to treat with a very small light radiation spot size using a quick coagulation process, very shallow vascular lesions enriched with typical micron size diameter blood vessels. These vessels are located mainly in very close proximity to the skin surface and have a small diameter red skin appearance with high concentration area of minor blood vessels. The invention device will therefore typically have a lesion size matching treatment exposure spot having a typical diameter of 4 mm and will be operated at a very slow pulse rate such as one illumination spot pulse in every 0.3-2 seconds and exposure of the lesion affected area for 3-5 repetitive exposure.

The most selective blood vessel thermolysis will occur when exposure time is less than the characteristic thermal conduction time constant of this target vessel. Exposure times significantly shorter than this may result in a mechanical fast expansion damaging process, while significantly longer exposures may result in harmful heat transfer to the surroundings epidermis tissue.

It is assumed here that a temperature rise of at least 42° C. is required, representing an increase from 38° C. to 80° C. Tissue structures will undergo coagulative necrosis at this temperature for millisecond-domain exposures.

It is further assumed that melanin is uniformly distributed across the follicular dimension and that any light impinging on the follicle at the irradiating wavelength (in the region 500-1000 nm) will be completely absorbed in a uniform fashion across the dimension. It is assumed that 20% of the incident energy from each exposure pulse within a (nominal) grid of contiguous 3 mm spots is absorbed in the lesion blood vessels.

In accordance with another embodiment this invention provides a system and a method for the combined treatment of Acne inflammatory lesions and for the reduction of the lesion localized redness.

The invention treatment method is enabled by a combined treatment effect consisting of destroying the P. acne bacteria by a violet/blue light exposure and selectively destroying small diameter blood vessels contained at a selected depth and in selected areas of a patient's dermis preferably in the vicinity of the acne lesion.

The present invention system also relates to a treatment device which is capable of treating post acne skin redness on small targeted areas situated on but not limited to the skin of the patient face, implementing a dedicated intense pulse light source, thus without affecting the surrounding of the post acne lesion and avoiding the creation of unwanted damages on the facial skin, such as the unwanted removal of hair from a male face.

The use of the invention system that involves the steps of (a) illuminating the acne affected skin area with a high intensity pulsed or non pulsed time gated light energy from a light source containing at least one spectral band of 400-450 nm; and (b) to deliver pulsed light to the area, the light in this step has at least one spectral band between 450 nm to 800 nm.

The use of the system wherein the steps of positioning the light source and operating the light source may be repeated in time intervals of several hours to several days until all such minor blood vessels creating the redness spots have been destroyed.

The present invention device preferably includes a pulsed light source that has at least one spectral band between 450 nm to 800 nm, and preferably;

(a) each light pulse delivering a fluence at the epidermis surface being treated of between 3 J/cm² and 6 J/cm²; and

(b) each pulse having a pulse duration of between 0.2 milliseconds and 2 milliseconds; and

(c) the exposed skin surface area may be between 0.01 square centimeters and 0.3 square centimeters; and

(d) the pulsed light source may be operated to deliver a single pulse, or multiple pulses may be provided; and

(e) the light source is selected from the group comprises a first light source that is continuous-wave light source with pulse duration being controlled by gating the light source to provide the pulsed light and a second light source that is a slow repetition rate pulsed source with rate in the range of 0.3 to 2 Hz. The steps of positioning the light source and operating the light source may be repeated in time intervals of several hours to several days until all such minor blood vessels creating the redness spots have been destroyed.

According to one embodiment of the present invention there is provided a method of treating acne by the generation and radiation on acne affected skin of non-coherent high intensity light energy, combined of: (a) a violet/blue active light source for the eradication of P. acne bacteria; and (b) generation of yellow-rich broad spectrum non coherent light having a short pulse duration for the treatment of post acne redness of the skin mainly localized the acne lesions area.

According to another embodiment of the present invention there is provided a device which eliminates by minor veins coagulation in the skin red spots present after the treatment of inflammatory acne lesions, said device is designed to treat a small number of small local spots and not to cover by its radiation large surface areas of the treated skin. Wherein preferably; (a) said system utilizes an intense pulsed light source which is rich with yellow spectrum light; (b) said device irradiation spot size is less then 6 mm in diameter fitted for the treatment demands of residual post acne lesion skin area redness; and (c) said device is designed to operate at a low pulse rate of less than 2 PPS and for irradiating up to 20 seconds on each spot.

According to yet another embodiment of the present invention there is provided an acne integrated treatment system consisting of: (a) said device; and (b) an intense blue/violet light source.

According to yet another embodiment of the present invention there is provided an acne integrated treatment system wherein the blue/violet light source is used for the eradication of P. acne bacteria within the inflammatory acne lesion and on the skin and for the reduction of inflammation and an integrated device of intense pulse light rich with yellow light, is used for the treatment of residual lesion area redness.

According to yet another embodiment of the present invention device, a preferred device is constructed from: (a) a Xenon flash light source with very short arc length and up to 500 microseconds pulse duration; (b) an optical assembly which enables the collection and focusing of the light from the light source on the treated skin with energy spot smaller than 4 mm and (c) the energy of the spot is high enough energy to coagulate blood and contract 15-50 microns size blood capillaries within a few seconds.

According to yet another embodiment of the present invention device the optical assembly for controlling the treatment energy spot size and shape is constructed from at least one optical element selected from the optical elements group including a beam shaping and concentrating light guide, a focusing optical module and an optical mask at the exit aperture of the device.

According to yet another embodiment of the present invention device, the device has large exposure area enhanced energy and short pulse duration thus enabling the removal of hairs or affecting skin in the vicinity of the post acne lesion; and with the integration of an optical mask at the exit aperture of the device, wherein the mask has an aperture in the typical size of a post acne lesion, than the device energy exposure is limited to local lesion redness reduction while eliminating the capability of removing hairs or affecting surrounding skin when mask is not presented.

The foregoing and other objects, features and advantages of the invention will be apparent from the following more particular description of preferred embodiments of the invention as illustrated in the accompanying drawings.

### BRIEF DESCRIPTION OF THE DRAWINGS

The invention is herein described, by way of example only, with reference to the accompanying drawings, wherein:

FIG. 1 is a schematic front and side view illustrations of one embodiment of the photodynamic treatment apparatus according to the present invention.

FIGS. 2A and 2B are schematic side view and front view illustrations respectively of the illumination head unit, the same embodiment of the present invention apparatus wherein the illumination unit head structure is based on a violet/blue light source of a gas discharge lamp;

FIGS. 3A and 3B are schematic top and bottom views respectively of the light source unit in the apparatus of FIG. 1, in an embodiment wherein the illumination unit structure is based on a circular array of LED's, or laser diodes, emitting a narrow spectral band red light illumination component, the array is integrated on the perimeter of a parabolic cross-section reflector, in the focal point of which is situated a high illumination intensity, narrow spectral band, violet/blue light gas discharge light source;

FIG. 4 is a schematic bottom view illustration of the present invention violet/blue light source, in another embodiment, wherein the illumination unit structure is based on a two dimensional array of LED's, or laser diodes, emitting a preferred narrow spectral band violet/blue light illumination component, the two dimensional array can also include any spatial distribution combination of violet/blue narrow spectral band emitting laser diodes or LED's, together with red light LED's, or laser diodes emitting in the preferred red spectral band;

FIG. 5 illustrates a typical spectral distribution of the light energy emitted by the present invention dedicated violet/blue light source, in the embodiments wherein the light source is a gas discharge lamp;

FIGS. 6A-6C illustrate another set of an additional three preferred embodiments of the illumination head in the apparatus according to the present invention, wherein all these embodiments are based on the application of a single axis elliptical cross-section cylindrical reflector, in the first focal point of which is fitted the illuminating gas discharge lamp arc. The image of the gas discharge light source arc is created in the second focal point of the elliptical reflector and can be then directly used for object illumination, or collected and further conducted by a fiber optic slit to circular beam shaping bundle, or collected and reshaped by a dedicated set of two orthogonal cylindrical lenses, to optimally conduct and collimate the light energy on the patient's treated skin areas;

FIG. 7 illustrates the results of the proposed apparatus, operated under laboratory controlled tests on p. acne, showing a decrease in propionibacterium acnes in 4-5 orders of magnitude, after two 30, or 60 minutes exposures separated by 72 hours of dark incubation;

FIGS. 8A and 8B are two schematic views illustrations of another embodiment the present invention light source apparatus, wherein in FIG. 8A the illumination source is operated through a single illumination head;

FIGS. 9A and 9B are two schematic views illustrations of another embodiment the present invention light source apparatus, wherein in FIG. 9A the illumination unit is structured of a dual illumination head configuration;

FIGS. 10A-10F are schematic side views illustrations of another embodiment of the present invention light source apparatus, wherein in FIG. 10A the illumination head is structured of an integrated dual illumination source;

FIG. 11B is a close look of the computerized control panel in FIG. 10A;

FIG. 11A is a back-side view of the present invention apparatus;

FIGS. 12 & 13 are graphical illustrations of the test results on the reduction in the number of lesions in the treatment of p. acnes using the apparatus of FIG. 1;

FIG. 14 demonstrates the results of the treatment by the apparatus of FIG. 1 for acne lesion reduction rate of over 60% in average, within a period of 4 weeks, versus other treatments results;

FIG. 15A is a cross-sectional view of an apparatus in accordance with the present invention for the treatment of acne and conditions and symptoms associated therewith;

FIG. 15B is a cross-sectional view of a further embodiment of the present invention for the treatment of acne and conditions and symptoms associated therewith;

FIG. 16 is a schematic view of an embodiment of the apparatus of the invention employing an intense pulsed light generating device;

FIG. 17 is a schematic view of another embodiment of the apparatus of the invention employing an intense pulsed light generating device;

FIG. 18 is a cross-sectional view of an embodiment of the present invention for treating acne and conditions and symptoms associated therewith including inflammation and redness wherein the output spectrum can be controlled by changing a filter inserted into the illumination source optics;

FIGS. 19A-19C are perspective views of various embodiments of the present invention in place for the treatment of acne and conditions and symptoms associated therewith;

FIGS. 20A-20C are perspective views of further embodiments of the present invention for the treatment of acne and conditions and symptoms thereof;

FIGS. 21A-21D are cross-sectional view of embodiments of light sources suitable for spot size intense pulsed light treatment for use in the present invention;

FIG. 22 is a perspective view of an embodiment of the present invention for the treatment of acne and conditions and symptoms thereof; and

FIG. 23 is a perspective view of still another embodiment of the present invention for the treatment of acne and conditions and symptoms thereof.

### DESCRIPTION OF THE PREFERRED EMBODIMENTS

The present invention is an apparatus, a device and a system which can be used for phototherapy treatments of skin disorders. Specifically, the present invention can be used for the non-invasive treatment of acne vulgaris and seborrhea, and for the reduction of inflammation and skin redness in the acne affected skin areas, thereby enabling treating various parts of the patient's body with ability to control the illumination power, energy spatial distribution, exposure duration and illumination source emittance spectral bands.

The principles and operation of the apparatus for phototherapy treatment according to the present invention may be better understood with reference to the drawings and the accompanying description.

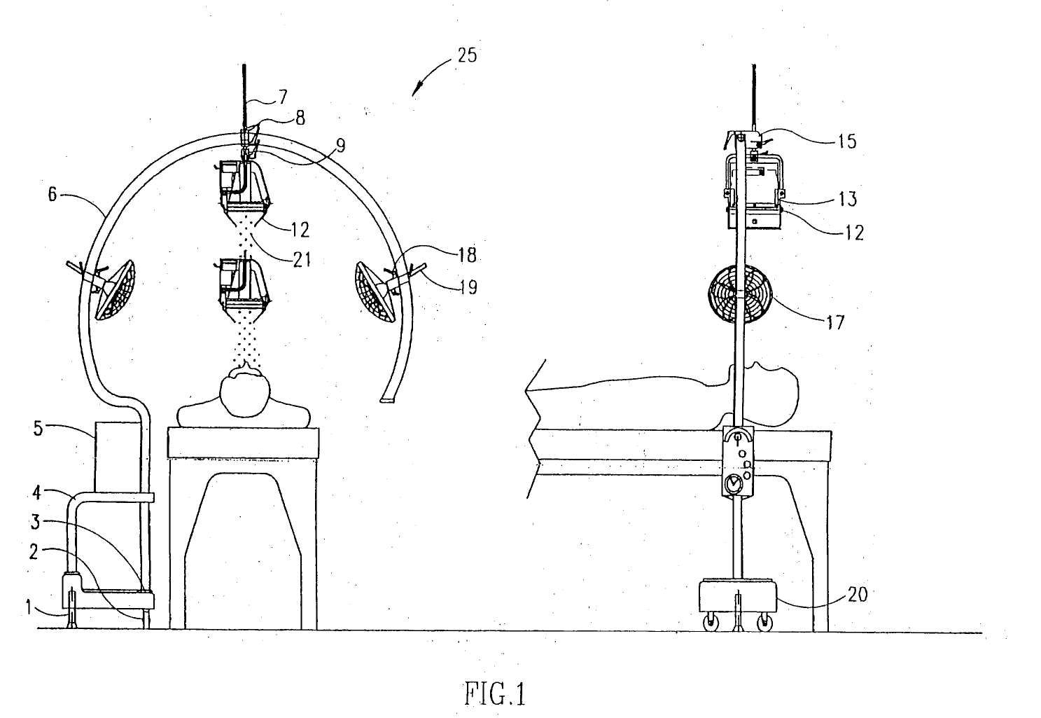

Referring now to the drawings, FIG. 1 is a schematic front and side view illustration of the photodynamic treatment apparatus according to the present invention, which is referred to herein below as system 25.

System 25 includes a violet/blue light source fixture 13, which can be moved repositioned and directed to the treated patient specific skin area by adjustment unit 15. It can also be lifted up or lowered down in order to change the effective radiated energy flux on the treated area, by using pole unit 7 and handle 8. The apparatus light source is mounted on a mechanical arc shaped fixture 6 for holding and supporting the light source at an adjustable distance and direction relative to the patient's treated skin area. The apparatus mechanical fixture 9 allows horizontal, vertical and radial placement and directing of light beam 21 from the light unit 13 to the patient's treated part of the body.

The high energy output, the consequent effective radiation flux and the relatively long distance of the treatment light source from the treated skin areas (typically in the order of 20-40 cm), is facilitated by the above described light source fixture 13. This apparatus light source fixture 13 thus enables an effective large skin area treatment in a single exposure, while typically using a fixed position for the light source fixture 13 during the entire treatment session of large skin areas, or entire human organs, as is clearly illustrated in FIG. 1.

Unit 17 is a schematic presentation of an air blower or a fan that serves to cool and remove access heat from the treated skin area. Units 18 and 19 are mechanisms to adjust the required position of unit 17.

Unit 17 is a schematic presentation of an air blower or a fan that serves to cool and remove access heat from the treated skin area. Units 18 and 19 are mechanisms to adjust the required position of unit 17.

Unit 5 is a control board for the apparatus enabling control of lamp power, illumination duration, air cooling operational parameters and general on/off and mains control functions.

Units 4 and 20 are a structural element and a balancing weight to stabilize the apparatus in a vertical up-right position. Unit 3 is a mechanical axis around which the entire apparatus arc shaped structure 6 can be rotated and refitted in any required horizontal angular position related to the treated patient bed 22.

Wheel 2 and pole 1 are elements required to move and refit the position of the apparatus according to the operational needs of the system operator.

Light source fixture 13 of consists of a lamp or a laser light source that emits violet/blue light with a peak at 405-420 nm. Close to a hundred percent of the light source ultra violet/blue light (UV) is filtered out by an integrated optical system. In a preferred embodiment of the light fixture 13, the illumination flux of the light fixture in the UV spectrum is less than 0.1 microwatt/cm² in the spectral range of 200-400 nm, to comply with UV free safety level requirements.

The required narrow spectral emission band of violet/blue light source is radiated by the present patent dedicated arc lamp due to a special gas mixture as described below, within the lamp, or by a gas laser source, or by a violet/blue light emitting semiconductor diode junction. The above light sources in a single source type embodiment, or in a combination of two or three type of light sources, allows optimal violet/blue light radiation with or without additional narrow spectral band lines in the red or green parts of the spectrum. The present invention light source enables the minimization of heat production at the treated target to a max. of 23 degrees Celsius on the epidermis at 30-40 cm. A mechanical shutter 12 in front of the light source 13 may be used to exactly define the treated area.

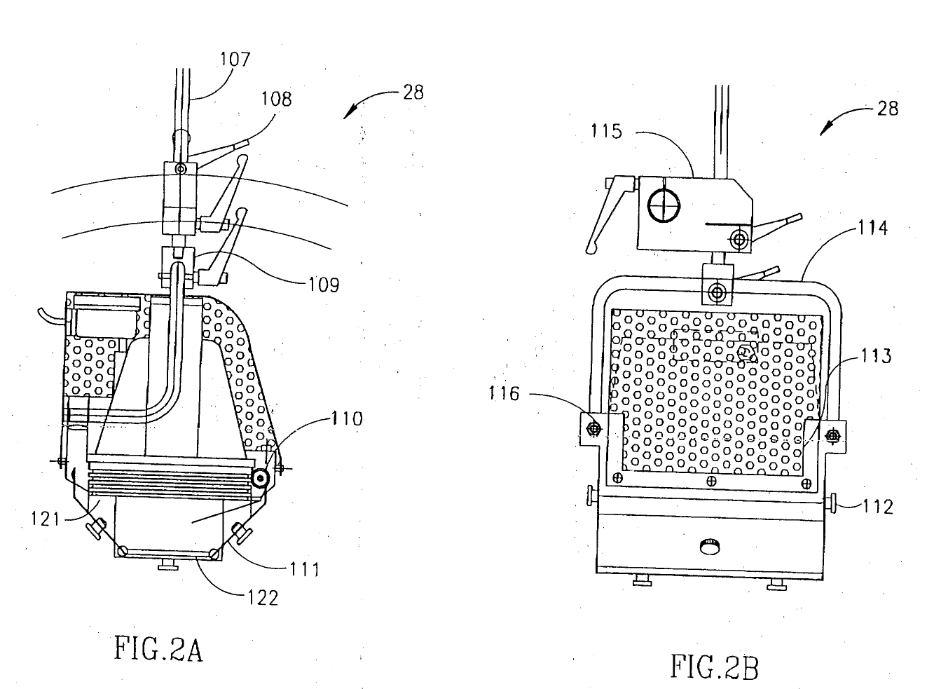

FIGS. 2A and 2B are schematic side and front view illustrations of the illumination head unit 13 according to the present invention, referred to herein below as system 28.

Illumination system 28 includes a filter unit 121 for filtering out the radiated energy spectral part, which is out of the preferred specific bandwidth in the violet/blue and/or the red spectrum, as previously described in the above background paragraph of the invention. Unit 111 is a set of four mechanical flaps with a control knob 112 and a pivoting axis 110 that create together an adjustable aperture iris unit to control the size and collimation parameters of system 28 radiated light beam. U shaped arm 114 holds and supports the illumination unit housing 113. Unit 109 enables rotation of the system 28 around vertical pivot axis 107 and to lock it in the preferred rotational angle. Unit 115 enables changing position by sliding and further fixing in a preferred position system 28 along the apparatus support arc 106. Unit 115 also enables sliding system 28 up or down and then fixing its position. Unit 122 is an optional mechanical support housing and a lens for focusing and concentrating the system 28 illumination beam on a smaller area of the treated skin, thus creating a higher light energy flux whenever required for a specific treatment.

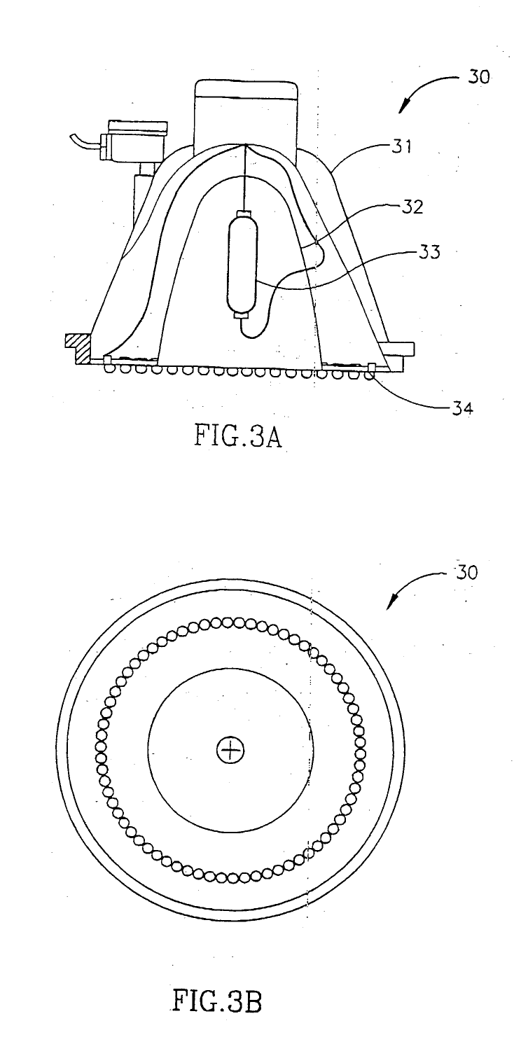

FIGS. 3A and 3B are schematic top and bottom views of another preferred embodiment of the present invention lighting head unit 13 of the apparatus described in FIG. 1, referred to herein below as system 30.

System 30 includes a housing unit 31 and a reflector 32 having preferably a parabolic vertical cross section. The gas discharge lamp, or the Excimer lamp 33 is assembled into reflector unit 32 in a way that fixes the center of the lamp illumination arc in the focal point of the reflector. Lamp 33 may be a specially designed Gallium and Lead halides gas mixture discharge lamp with peak emission in the 405-430 spectral band, or a dedicated Excimer lamp with peak emission in the spectral range of 400-430 nm.

Unit 34 is a circular array of red emission LED's or red light laser diodes installed around the aperture perimeter of the reflector unit 33.

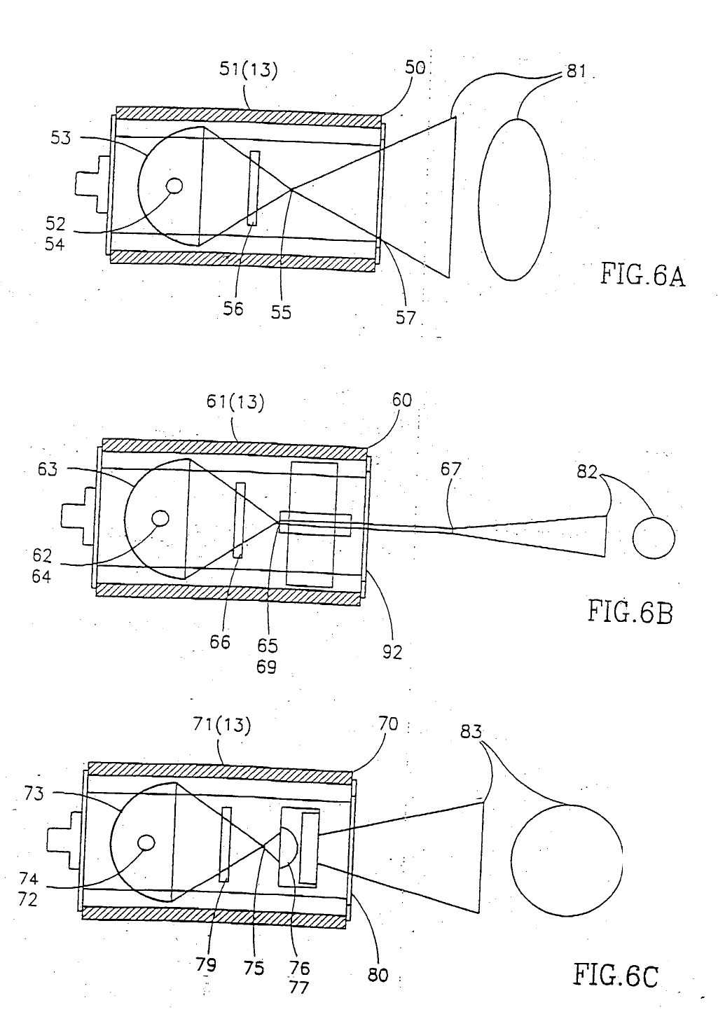

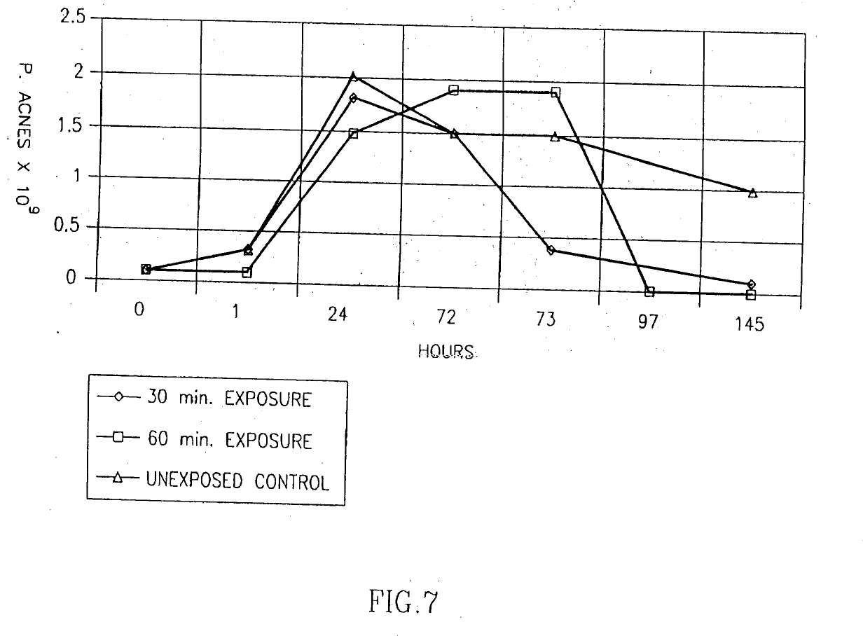

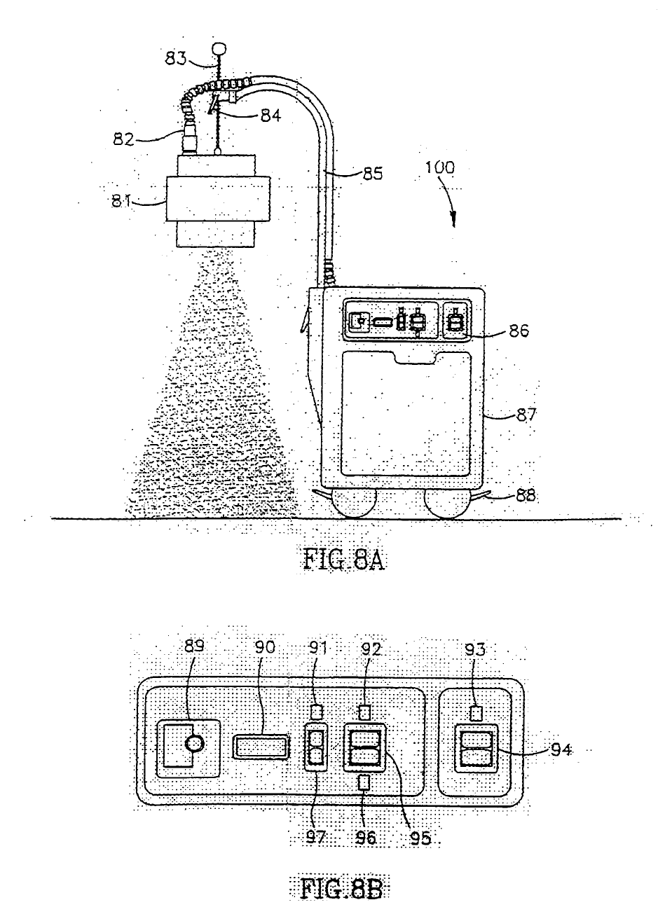

The typical effective illumination output energy of most industrial types of metal-halogen gas-discharge lamps and Excimer lamps is relatively high and is in the order of tens to hundreds of Watts. This energy level of the light radiating source, when properly collected and directed by the optical reflector 32 in this embodiment, or by other alternative types of optical light collection and direction sub-systems, such as, but not limited the configuration of the optical sub-system composed of reflector 73 and lenses 76 and 77 of FIG. 6C following, can generate an energy output flux having a typical flux value variable in the range of 10-500 mw/cm square on any spot in the illuminated skin area of the treated body. This energy flux range is high enough to facilitate an effective photo-therapeutic effect on an entire treated human limb or body part, typically over 400 cm square in area.

The Excimer lamps are produced by Heraus Nobellight from Hanau, Germany, Rhombic Corporation from the USA and Quark Ltd; they all use a highly efficient photon emission reaction (7-50%) from excimers to produce wavelengths of vacuum ultra violet (VUV) to visible light. An excimer is an excited state in a molecule that dissociates into an unbound state. This feature means that self-absorption in the lamp is small, and because of this, the lamp can be scaled to large volumes without severe degradation of the emission wavelength. The pioneering excimer lamp technology developed by Columbia Research Instruments is now owned by Rhombic Corp. It efficiently transfers the energy of electricity to microwaves and microwaves to excimers (transfer efficiency between 50 and 90%). This technology produces light of a pure wavelength more efficiently than any other light source. This is significant because light is used to induce chemical and photobiological reactions that are wavelength specific. The excimer lamp is orders of magnitude more cost effective (dollar/photon bandwidth) than anything that current technology is capable of producing.

State of the art other UV to visible light sources include lamps like mercury, xenon, argon, tungsten halogen, fluorescent, incandescent and arc lamps and in parallel various types of lasers. As a concept these competitors are not capable of producing high efficiency narrow band excimer radiation. For all other technology based light sources the photons that are beyond the range of a chemical reaction's requirements are wasted. Rhombic was a pioneer in excimer lamps, companies such as Heraeus Noble Light, and Quark Ltd., have since come into the market.

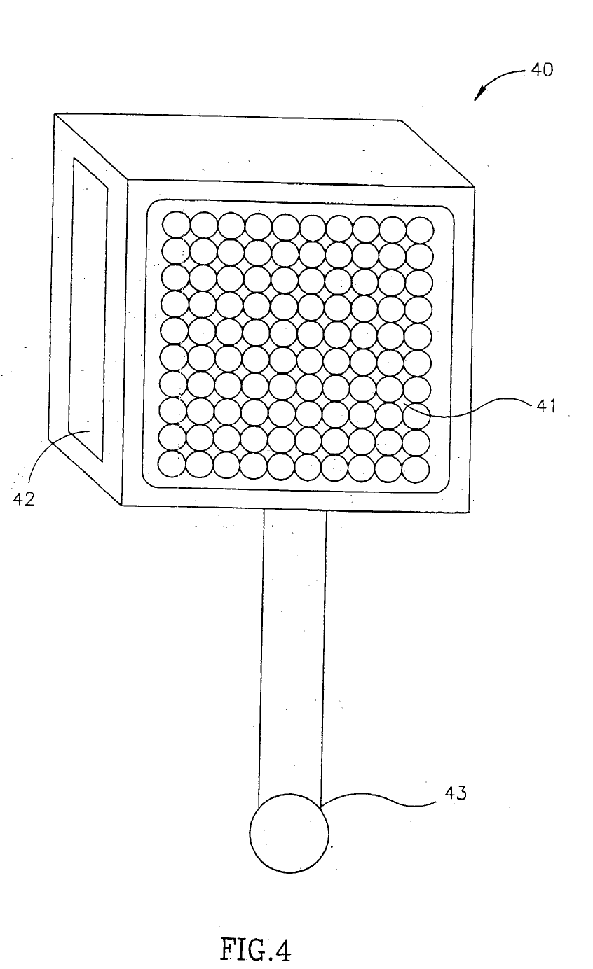

FIG. 4 is a schematic illustration of another preferred embodiment of the present invention lighting head unit 13 of the apparatus described in FIG. 1, referred to herein below as system 40.

System 40 includes a housing unit 42 and a two-dimensional array of LED's, or laser diodes 41, emitting a narrow spectral band violet/blue light illumination component. These semiconductor solid state light sources can be GaN or ZnSe components. The two-dimensional array can also include narrow spectral band red light LED's, or laser diodes, emitting in the preferred red spectral band. Unit 43 is a mechanical structure for attaching system 40 to the apparatus of FIG. 1.

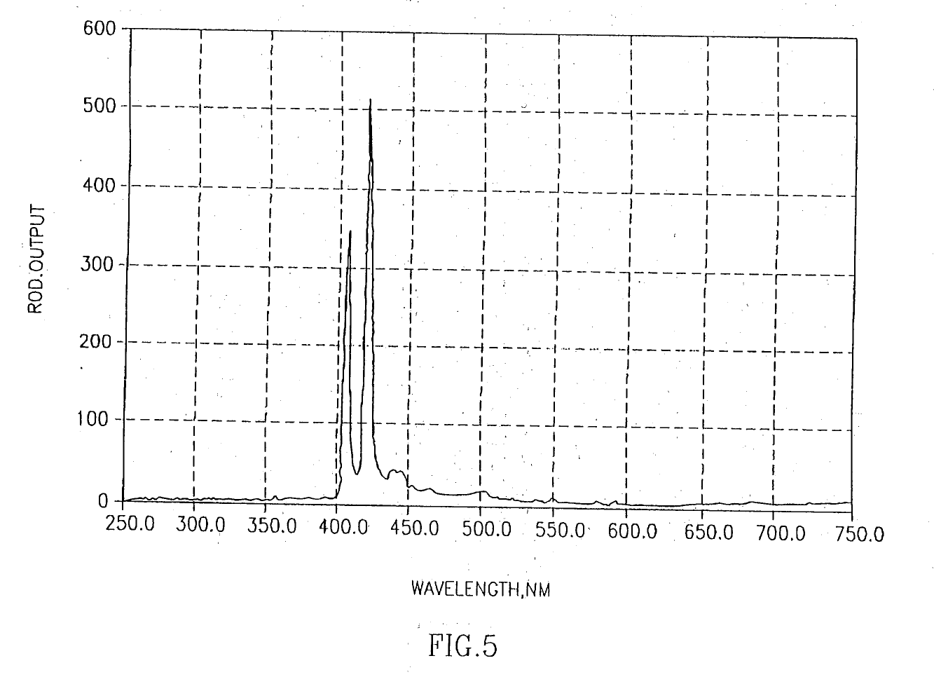

FIG. 5 illustrates a typical spectral distribution of the light energy emitted by the present invention dedicated violet/blue gas discharge lamp based light source embodiment, before further spectral optical filtration is done, in the embodiments wherein the light source is a gas discharge lamp.

FIG. 6A is a schematic cross section illustration of one of a set of three possible preferred embodiments of the present invention lighting head unit 13 of the apparatus described in FIG. 1, the first possible embodiment is referred to herein below as system 50. Light source head embodiment of system 50 consists of a housing 51 that supports an arc lamp, or an Excimer lamp, or a line beam shape laser light source 52 that emits violet/blue light with a peak at 405-420 nm. The light source is fixed in the first focal point 54 of an elliptical cross section shape reflector 53. The energy emitted out of the preferred spectral band reflected by the elliptical shaped reflector and is imaged as a line source at its second focal point 55. From the secondary focal point the beam is diverging at a small angle and creates an oval shaped illumination area 81 of typical size 20×10 cm. at a convenient treatment distance of 40 cm. from the lamp housing exit aperture. The non violet spectral part of the light source emission is rejected and filtered out by filter unit 56 and the lamp housing is sealed by tempered glass window 57 possibly coated with a heat mirror layer for the protection of the patient against heat and explosion. The required narrow spectral emission band of violet/blue light source is radiated by the present invention dedicated arc lamp due to a special gas mixture within the lamp, or by a violet/blue light emitting semiconductor diode junction array. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

FIG. 6B is a schematic cross section illustration of a second possible preferred embodiment of the present invention lighting head unit 13 of the apparatus described in FIG. 1, the second possible embodiment is referred to herein below as system 60. Light source head embodiment of system 60 consists of a housing 61 that supports an arc lamp, or a line beam shape laser light source 62 that emits violet/blue light with a peak at 405-420 nm. The light source is fixed in the first focal point 64 of an elliptical cross section shape reflector 63. The energy emitted out of the preferred spectral band reflected by the elliptical shaped reflector and is imaged as a line source at its second focal point 65. In the secondary focal point the beam enters a slit shape fiber bundle aperture, matching the size and shape of the imaged light line at this point 68. At the exit circular aperture 67 of this fiber bundle the emerging light is diverging at a typical 40 degrees angle and creates a circular shaped illumination area while its size and consequently the illumination power density can be controlled by changing the distance from the exit fiber end 67 to the patient treated skin area. The non-violet spectral part of the light source emission is rejected and filtered out by filter unit 66 and the lamp housing is sealed by a cover window 69. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

FIG. 6C is a schematic cross section illustration of a third possible preferred embodiment of the present invention lighting head unit 13 of the apparatus described in FIG. 1, the third possible embodiment is referred to herein below as system 70. Light source head embodiment of system 70 consists of a housing 71 that supports an arc lamp, or a line beam shape laser light source 72 that emits violet/blue light with a peak at 405-420 nm. The light source is fixed in the first focal point 74 of an elliptical cross section shape reflector 73. The energy emitted out of the preferred spectral band reflected by the elliptical shaped reflector and is imaged as a line source at its second focal point 75. After passing through in the secondary focal point 75 the beam is entering a set of two cylindrical lenses 76 and 77, which are orthogonal oriented with respect to their linear axis. At the exit of this lens system aperture 78 a close to a circular light illumination area is created of typical size 20×20 cm. at a convenient treatment distance of 40 cm. from the lamp housing exit aperture. The non violet spectral part of the light source emission is rejected and filtered out by filter unit 79 and the lamp housing is sealed by a cover window 80. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

The method according to the present invention improves the results by adding oxygen transporting compounds based on perfluorocarbons and/or oxidative and/or keratolytic agent, daily and or immediately pretreatment. The proposed oxygen transporting agents i.e., perfluorocarbons lipophilic emulsion, release nascent oxygen directly into the sebaceous glands achieving a 76% O₂ environment as compared to the atmospheric 20%. The proposed oxidative agents i.e., emulsion or gel of H₂O₂ 1-10%, release by contact with the enzyme cathalase present in the skin nascent oxygen. The specific formulations of the emulsion or gel prevent the upward release of the oxygen and cause a short temporary inward pressure of up to 15 Atm. of O₂, penetrating to the sebaceous glands situated in the deeper layers of the skin.

The oxygenation of the skin during the phototherapy process raises the efficiency of the desired photodestruction of p. acnes and thus decreases of acne lesion number and severity. Added keratolytic agent (i.e. 1-5% salicylic acid) to the applied formulation will enhance diffusion of O₂ into the sebaceous glands. Cooling of the applied emulsion or gel minimizes the heat in the epidermis thus allowing a further increase of the light intensity in the sebaceous glands.

FIGS. 8A and 8B are illustrations of two schematic views of another embodiment the present invention violet/blue light source apparatus 100, wherein in FIG. 8A the apparatus 100 illumination source is installed within and operated from a single illumination head 81. The illumination head 81 is operated by a power supply and electronic control unit 87 and is supported by an adjustable height supporting mechanism 85. Lever 84 enables further fine adjustments of the lamp head 81 distance from the treated area, by sliding up or down and tightening at the requested position the support pole 83. Cable harness 82 connects the illumination head 81 to the power supply and electronic control unit 87. Control panel 86 enables the operation and control of the operational parameters of the power supply and electronic control unit 87. Unit 87 is supported by a set of four maneuvering wheels 88, having an integrated stop and fix in place mechanism.

FIG. 8B is a close look of control panel 86 in FIG. 8A. 89 is an electronic timing mechanism for controlling the treatment time. Counter 90 is a time-laps numerical indicator, for counting the accumulated operational hours of the illumination head 81. Switch 97 and indication lamp 91 control the operation of a fan cooling module, integrated in the illumination head, having the task of cooling the slightly heated illuminated skin area, during the treatment time. Switch 95 and indication lamps 92 and 96 enable switching and selecting the intensity of illumination between two discrete pre-selected energy levels. Switch 94 and the attached status indication lamp 93 is the system main power switch.

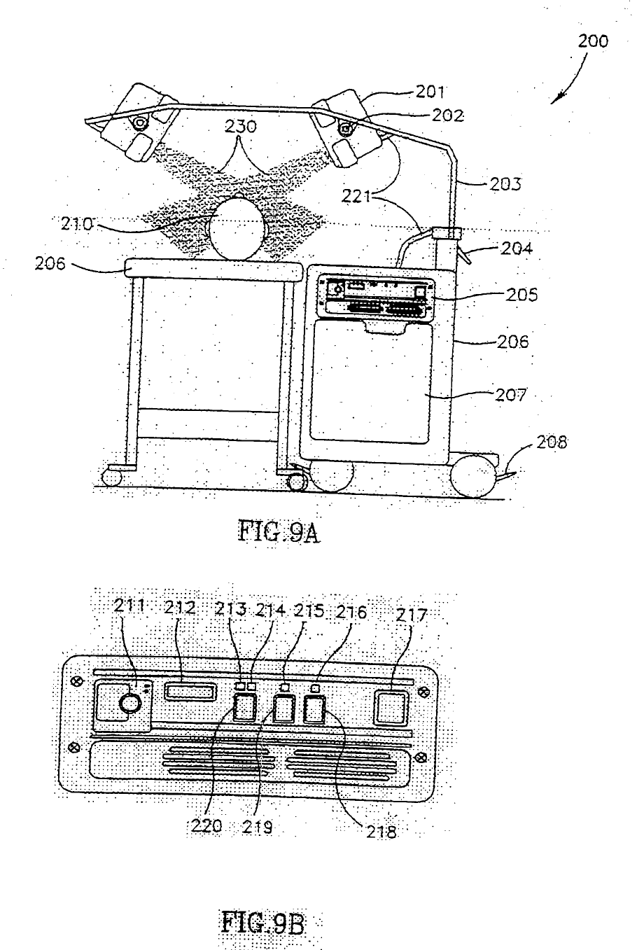

FIGS. 9A and 9B are two schematic views illustrations of another embodiment the present invention violet/blue light source apparatus 200, wherein in FIG. 9A the illumination source is structured of a dual illumination head module 201. The dual illumination head module 201 is operated by an integrated power supply and electronic control unit 206 and is supported by an adjustable height supporting mechanism 203. The head 210 height positioning related to the treated area 206 is done by sliding up or down a supporting pole with an integrated piston unit which is a part of the support mechanism 203 and then tightening the lever 204 at the requested height. Cable harness 221 connects the illumination heads 201 to the power supply and electronic control unit 206. Control panel 205 enables the operation and control of the operational parameters of the power supply and electronic control unit 206. Unit 206 is supported by a set of four maneuvering wheels 208, having an integrated stop and lock mechanism. The two illumination heads can slightly vertically tilted by the operator around pivot axis 202, in order to adjust the positioning and consequentially the illumination energy distribution of the two illumination collimated light beams 230, to be equally and evenly distributed on the two face sides of the treated patient 210.

FIG. 9B is a close look of control panel 205 in FIG. 9A. 211 is an electronic timing mechanism for controlling the treatment time. Counter 212 is a time-laps numerical indicator, for counting the accumulated operational hours of the dual beam illumination head module 201. Switch 220 and indication lamps 213 and 214 enable treatment duration control through timer 211 in one switch position, or unlimited operation time, by switching to the other switch position. Switch 219 and indication lamp 215 control the operation of a fan cooling module, integrated in the illumination head, having the task of cooling the slightly heated illuminated skin area of patient 210, during the treatment time. Switch 218 and indication lamps 216 enable switching and selecting the intensity of illumination between two discrete preselected energy levels. Switch 217 is the system self illuminated, main power switch.

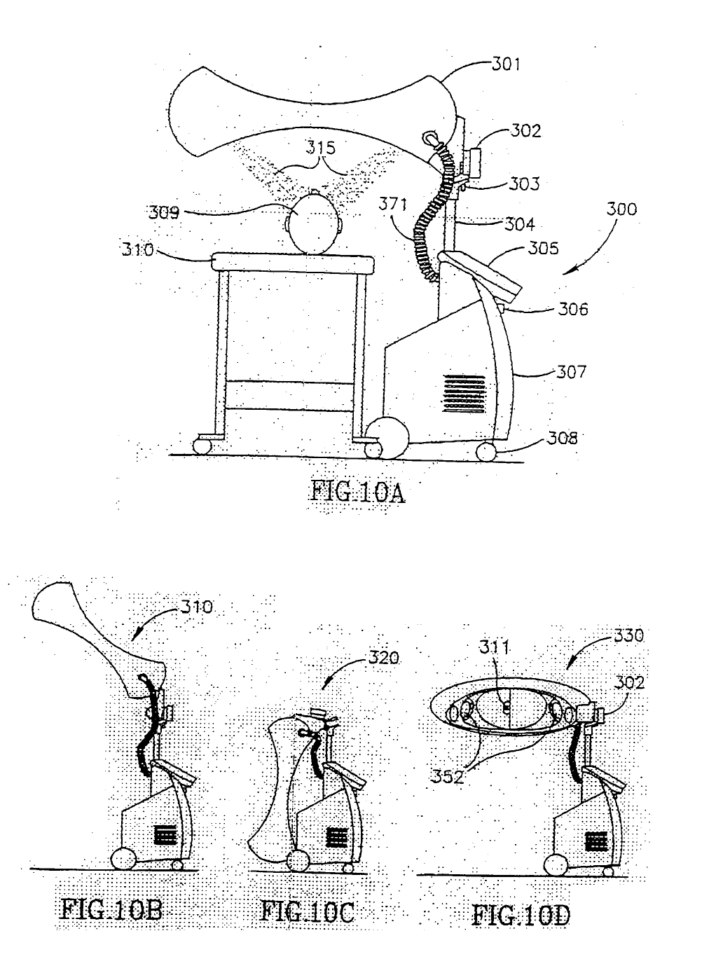

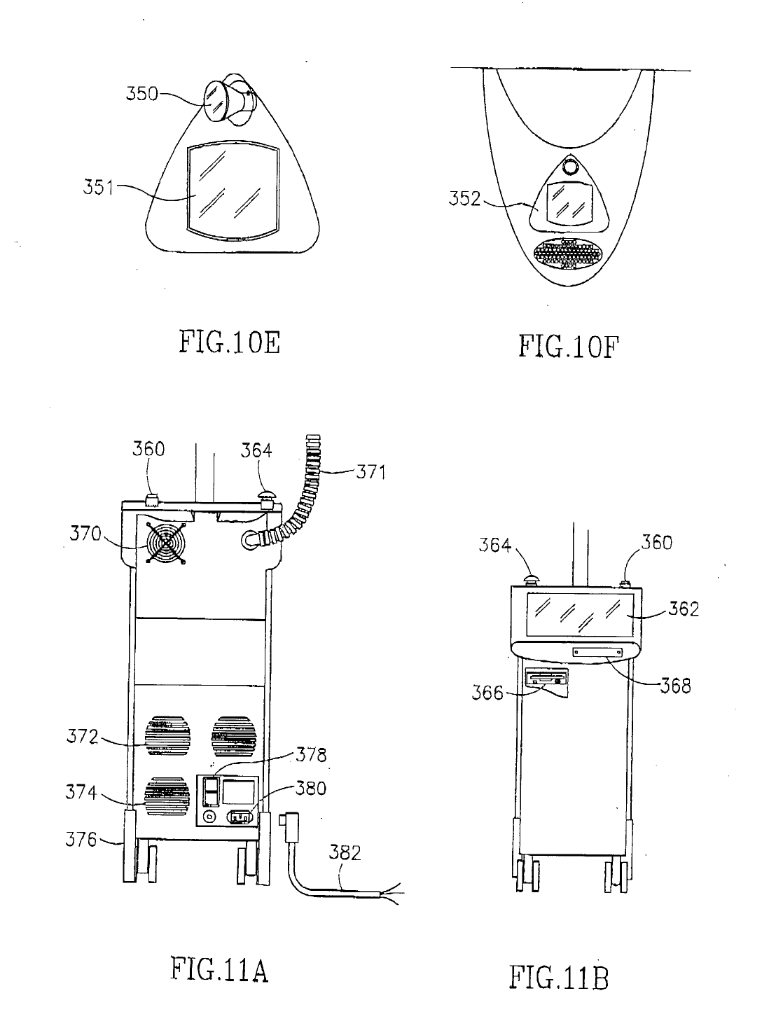

FIGS. 10A-10D are schematic side views illustrations of another embodiment of the present invention violet/blue light source apparatus 300, wherein in FIG. 10A the illumination source is an integrated dual illumination source head 301. The dual illumination source head module 301 is operated by a power supply and electronic control unit 307 and is supported by an adjustable height, supporting mechanism 304. The adjustment of the lamp heads unit 301 distance from the treated area 310 and the patient treated skin area 309, is done by sliding up and down through the operation of an electro-mechanic piston to reach the requested position of the support pole 304. Cable harness 371 connects the illumination heads 301 to the power supply and electronic control unit 307. Digital video-graphic display control panel 305 enables the operation and control of all the operational parameters of the power supply, the electronic control and the computer modules of unit 307. Unit 307 is supported by a set of four maneuvering wheels 308, having an integrated stop and lock mechanism. The two illumination units inside the illumination head 301 can be slightly vertically tilted by the operator in order to adjust the positioning and consequently the illumination energy distribution of the two illumination collimated light beams 315, to be equally and evenly distributed on the two face sides of the treated patient 309.

FIG. 10B, is the second of the four schematic side views of the present invention apparatus, showing another optional functional position of the illumination head, illustrating the present invention violet/blue light apparatus embodiment of FIG. 10A.

In FIG. 10B, the illumination head folding optional position 310 enables the up-tilting of the illumination head to a position required for temporary clinic storage periods and for the patient better maneuverability after treatment session completed to support post-treatment quick patient release requirements.

FIG. 10C is the illustration of the apparatus 300 in position 320, wherein the head is folded down to minimize size and packing volume for long term storage and for packaging and transportation.

In FIG. 10D, the illumination head of the apparatus 300 is pivoted around axis 302, thus enabling the head swivel to support in any requested “roll” angle position, this feature is desired to enable treating of a patient in a seated or partially-lying position. FIG. 10D also illustrates the integrated imaging module of the apparatus 300. Imaging sequence is first done is done by a miniature digital video and sill camera 311, installed in the center of the illumination source head 301. The acquisition step of the patient treated skin area picture by camera 311, is followed by the digital image processing, analysis and related treatment progress parameters evaluation by the apparatus 300 integrated computer module.

FIGS. 10E and 10F is a close look of the illumination output-window aperture 352 of the illumination head 301 and on the ventilation air input duct aperture 354, of the illumination head 301. 350 is a halogen or tungsten filament lamp, geared for the illumination of the patient treated area, illumination is required for the optimal image condition during computer controlled pre treatment imaging. 351 is the illumination unit glass protected output aperture window.

FIG. 11B is a close look of the control panel 305 in FIG. 10A. 362 is a digital video-graphic display unit having preferably an overlaid touch screen unit that supports the registration and the X-Y positioning parameters of the operators touching point in any co-ordinate location on the screen. By touching the screen 362 on discrete points, when the treated skin area still image is displayed on the screen 362, the operator can electronically mark the affected areas, or points, on the treated skin area. The computer module of unit 307 can further accumulate the number and position information of the affected points and areas and further process this information to create and display on the apparatus screen 362 any required monitoring data regarding the healing effect progress from treatment to treatment. Switch 360 is the system main on-off switch that controls the system awakening and shutting-off processes through wire operating special commands on the system computer. 364 is a Panic switch that cuts-off the input power of the mains supply to the unit, in case of emergency. The computer module of unit 307 operates an adjustable electronic timing mechanism for controlling the treatment time. 306 is a Floppy diskette drive through which treatment data is downloaded from the system and back-up or revised software versions are uploaded to the apparatus 300 computer module.

FIG. 11A is a back-side view of system 300. 370 and 374 are ventilation units for the cooling of the computer and the electronic sub-units of the control unit 307. Switch 378 is the system main power/safety on-off circuit breaker switch. 372 are two loudspeakers for generating computer synthetic voice commands and instructions to the system operator and the treated patient. Cable harness 371 is connecting and conducting the power lines and the control commands from the electronic and computer unit 307 to the illumination head 310.Cable 382 is the mains supply cable and plug and 380 is the power input socket.

In Vitro Experimentation (Referring now to FIG. 7)

Photodestruction of p. acnes:

Bacterial strain. The strain used in this study was Propionibacterium acnes 6919 which was obtained from the American Type Culture Collection (ATCC) at Rockville, Md. U.S.A.

Growth media—Propionibacterium acnes was grown on Reinforced Clostridial Agar from Oxoid (Basingstoke, Hampshire England) at pH=6-6.2.

Illumination tests were carried out when bacteria were grown in Reinforced Clostridial Broth which was prepared from the same ingredients except the agar at pH 6-6.2.

Illumination method—Illumination was carried out by CureLight's acne therapy system. Under blue light at the wavelength of 407 nm Illumination periods were 15 minutes, 30 minutes or 60 minutes as indicated in the text Lamp intensity was 20 mW/cm².

Bacterial growth and illumination—Propionibacterium acnes was transferred from the bacterial stock into Reinforced Clostridial Agar Plates. Bacteria were streaked on the plated for isolation of single colonies by the “clock plate technique”. These plates were called “Start plates” and were incubated for three days under aerobic conditions in an anaerobic jar. The jar contained Aaero Gen sachets from Oxoid, England to maintain anaerobic conditions suitable for p. acnes.

From the “start plates” single colonies were transferred into Reinforced Clostridial Broth and allowed to grow anaerobically for 24 hours or for 72 hours. Bacteria grown for 24 hours were defined as “young culture” and those grown for 72 hours were defined as “old culture”. The “young” or the “old” cultures were transferred to illumination for the indicated times. Some cultures were illuminated again after 24 hours or 48 hours from the first illumination as indicated in the results. After each illumination a sample was taken out from the culture and viable bacteria were counted. Viable bacteria were monitored and calculated by counting the colony forming units after appropriate dilution in saline and cultivation on the Reinforced Clostridial Agar plates under anaerobic conditions for three days. The colony forming units of the survivals were calculated per ml. Untreated cultures served as controls.

Results have shown that exposure to the proposed apparatus achieves a decrease in propionibacterium acnes from 10⁹ to <10⁴ after two 30, 60 minutes exposures separated by 72 hours of dark incubation, as shown in FIG. 7.

In addition, the destruction of p. acnes may be further enhanced by adding methylene blue 0.5% to the broth prior to irradiation.

Extensive pre-clinical tests were performed at Bar-Ilan University—Natural sciences lab by Prof. Zvi Malik and Prof. Yeshayahu Nitzan.

Results:

Illumination of “young” cultures—Propionibacterium acnes which was grown on a “start plate” was transferred into Reinforced clostridial Broth and incubated for 24 hours. After this period two illumination courses of 30′ minutes each were carried out in an interval of 48 hours from the first to the second illumination. The results demonstrate a decrease of 1 order of magnitude in viability of the culture in comparison to the control. When the culture taken from the “start plate” was grown for 24 hours and illuminated twice, this time for 60 minutes each and 48 interval between the illuminations, two orders of magnitude decrease in viability were demonstrated.

Illumination of “old cultures”—Propionibacterium acnes which was grown on a “start plate” was transferred into Reinforced Clostridial Broth and incubated for 72 hours. Bacterial cultures were illuminated once for 15 minutes or once for 60 minutes. Illumination in both illumination periods resulted in a decrease in the cultures viability of one order of magnitude. In addition, illumination of the old culture for two times for 30 minutes in an interval of 24 hours resulted in the decrease in viability of two orders of magnitude. When the “old” culture is illuminated twice for 60 minutes at an interval of 24 hours a decrease of four orders of magnitude is demonstrated in their viability.

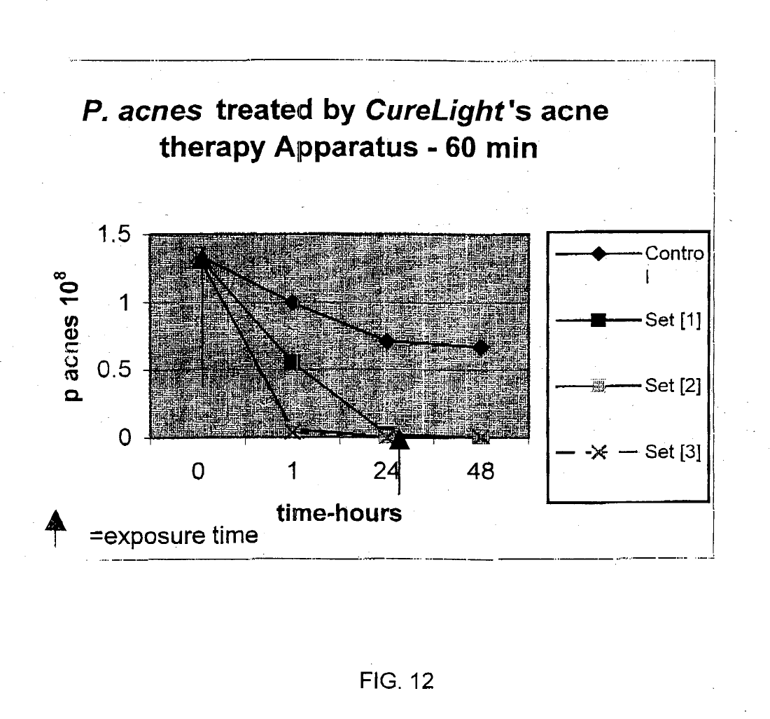

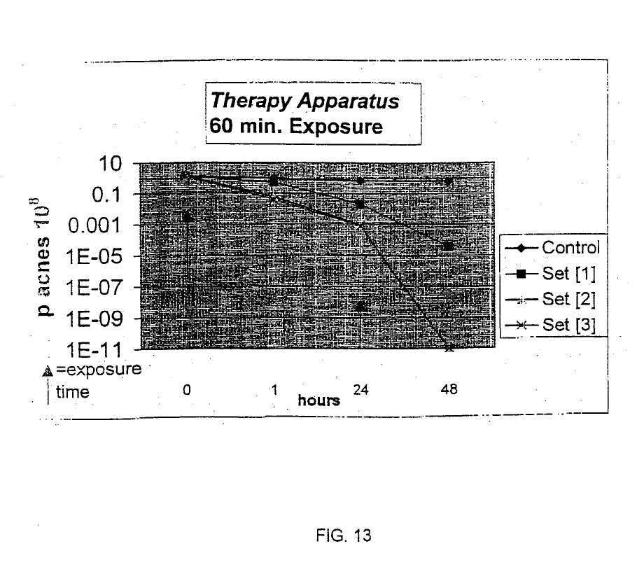

As shown in FIGS. 12 and 13, significant destruction of p. acnes was achieved up to 4 orders of magnitude.

The above laboratory experiments results prove that treatment of the acne bacteria with violet-blue light energy above a threshold energy level enables the destruction of the acne bacteria population to a much lower level, so as to get a significantly reduced post treatment bacteria population size. The invention apparatus energy flux supports bacteria destruction rates that surpasses the natural bacteria population average multiplication and growth rate between consecutive exposures, thus ensuring that the balance of the bacteria population will be significantly further reduced from one treatment to another, until total bacteria population reduction ratio will provide treatment results wherein post-treatment bacteria population will be at least 4 orders of magnitudes lower than the pretreatment bacteria population size.

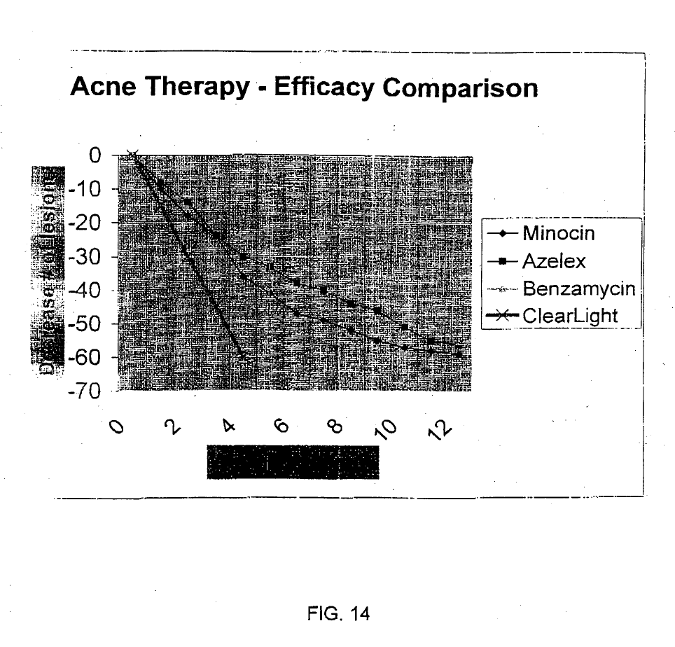

Clinical experiments have been performed on the population of over 100 patients in four clinical centers while using the invention apparatus. The results were compared to the state of the art acne lesion treatments. The results shown in FIG. 14 demonstrate an acne lesion reduction rate of over 60% in average in a period of 4 weeks, 2 treatments per week, of 15 minutes per treatment. These results should be compared to the same 60% reduction achieved in 12 weeks of daily treatments by prior art treatments.

In vitro Testing on Reduction of Pro Inflammatory Cytokines:

Inflammatory processes in the human body are regulated by a complex set of soluble molecules that are called cytokines. Some of the cytokines such as INF-gamma and TNF-alpha cause cutaneous and other human cells to produce and release molecules that directly cause inflammation. One of these PRO-inflammatory molecules is Interleukine-1 alpha (IL-1alpha).

It is known the Ultraviolet light is able to reduce induction of PRO inflammatory Cytokines. In another lab study we tried to find out if high intensity UV free blue visible light has also a direct anti inflammatory effect on human skin—unrelated to its ability to photodestruct acne bacteria.

We used two different cell lines: HaCaT (produced by spontaneous immortalization of genetically altered cell line) and hTERT (obtained by stable transfection of primary cell culture with hTERT resulting in expression of telomerase catalytic subunit and immortalization). Cell cultures were treated with INF-gamma and TNF-alpha and exposed to UVB light (310 nm at 50 mJ/cm², which is comparable to the dose used in treatment of some inflammatory skin conditions, such as psoriasis) and UV free visible blue light (405-440 nm at 90 mW/cm2. The expression of IL-1alpha and ICAM-1 was measured by quantitative ELISA at 48 hours. We found that exposure to high intensity blue visible light reduced the release of the above Pro-inflammatory cytokines by 50% and 60% respectively. This was similar to the effect caused by UV light in doses that are used in phototherapy of other skin diseases such as Atopic Dermatitis and Psoriasis (Reduced Pro-inflammatory cytokines by 70% and 80% respectively).

This in vitro study proves that high intensity blue visible light has a very significant direct anti-inflammatory effect on human cells. Its clinically proven high efficacy in Acne phototherapy is most probably based on both its photo destructive effect on acne bacteria thorough its porphyrines and on its direct anti-inflammatory effect on human keratinocytes. We use this in vitro discovery to achieve a rapid decrease of post acne inflammatory condition which still exists after the eradication of acne bacteria. This inflammatory condition was in prior art was addressed only after the eradication of p. acnes bacteria with the aid of topical or intralesional corticosteroids. High intensity blue visible light was not used for that purpose.

In addition to its anti inflammatory use on the acne sites the described cytokine reducing UV free narrow band high intensity blue visible light apparatus may serve as an efficient phototherapeutic tool for a spectra of inflammatory conditions such as skin ulcers, cutaneous autoimmune diseases such as Lupus Erythematosus, soft tissue and gums inflammation and Musculoskeletal and neuralgic traumatic and inflammatory conditions.

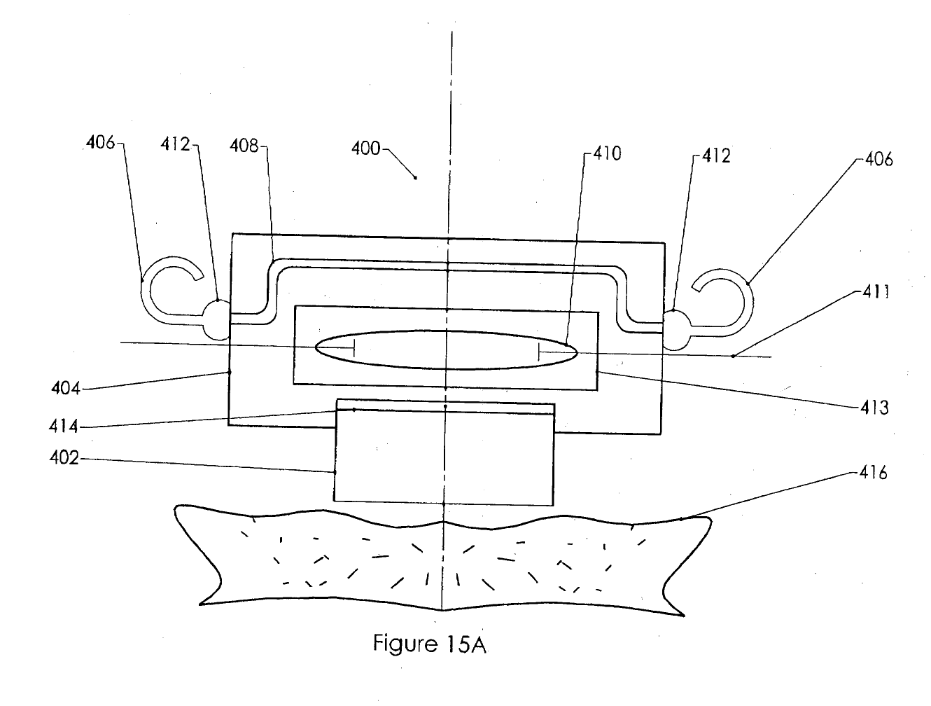

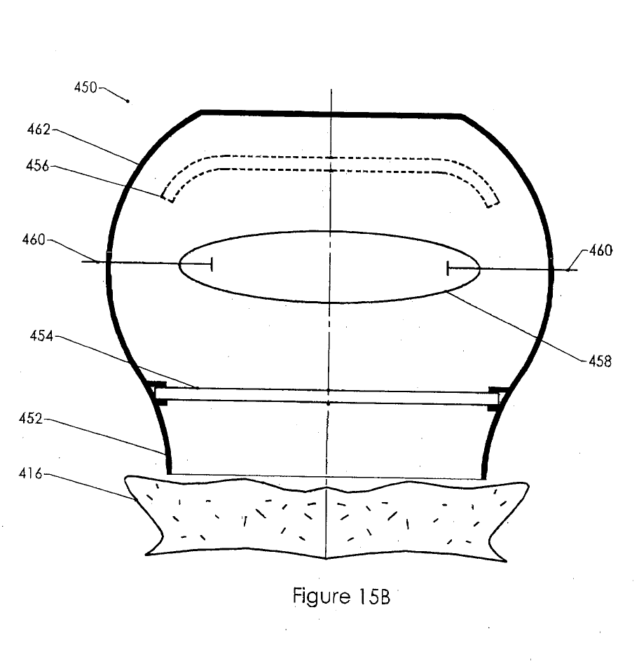

FIG. 15A illustrates a cross-section of an embodiment of the invention apparatus light source wherein the light is based on an intense light flash lamp with blue/violet (400-470 nm) enhanced light spectral output. This lamp is well adapted as the treatment light generator for the optimal treatment of acne seborrhea and inflammation. The light source 400 main elements are consisting of a tubular shape Xenon filler based flash lamp 410 and a light collection reflector 411.

The flash lamp can be supplied by Perkin Elmer, USA, or by Nobel Light-Heraeus GMBH, Germany. A blue/violet enhanced spectral content emission may be created by the introduction to the lamp glass or quartz tube internal volume of small quantities of Gallium, Indium and other rare earth metals, to act together with Xenon gas as the radiating active materials. A dedicated metal or ceramics housing 408 is integrating all the illumination source acting elements into a flash light based device 400. The device 400 is cooled by a liquid circulating cooler (not shown here) through feeding flexible pipes 406 and liquid pipe connectors 412. The apparatus output flash light is used for the treatment of acne lesions, seborrhea and inflammation on the patient's skin 416. The output of the flash lamp 410 is best collected by the cylindrical, parabolic or elliptical cross-section, reflector 411. The dedicated filter 414 may further filter the lamp energy output for the creation of over 25% violet/blue spectral content in the total output radiated emission of the device and the spectral filtering stage should be also keeping out all UV spectral content. The filtered light is energy is then further collected and conducted to the treated skin 416 by optical light guide 402.

FIG. 15B illustrates another treatment source embodiment of the invention treatment apparatus based on flash light according to the invention. A cross-section of an embodiment of the invention apparatus light source is demonstrated here wherein the light is also based on an intense light flash lamp with blue/violet (400-470 nm) enhanced light spectral output. This lamp is well adapted as the treatment light generator for the optimal treatment of acne seborrhea and inflammation. The light source 450 main elements are consisting of a tubular shape Xenon filler based flash lamp 460 and a light collection reflector 456.

The flash lamp of this embodiment may be also supplied by Perkin Elmer, USA, or by Nobel Light—Heraeus GMBH, Germany. A blue/violet enhanced spectral content emission may be created by the introduction to the lamp glass or quartz tube internal volume of small quantities of Gallium, Indium and other rare earth metals, to act together with or exclusively using Xenon gas as the radiating active materials. In this embodiment there are no liquid cooling elements and the heat energy created by the flash lamp is partly disposed off by ventilation or conduction and partly radiated towards the treated skin. A housing 462 is packaging all the illumination source elements into a flash light based apparatus light source module 450. The apparatus output radiated flash light is used for the treatment of acne lesions, seborrhea and inflammation on the patient's skin 416. The output of the flash lamp 410 is best collected by the cylindrical, parabolic or elliptical cross-section, reflector 456. The dedicated filter 454 may further filter the lamp energy output for the creation of over 25% violet/blue spectral content in the total output radiated emission of the device and the spectral filtering stage should be also keeping out all UV spectral content. The filtered light energy is then directly radiated towards the treated skin 416.

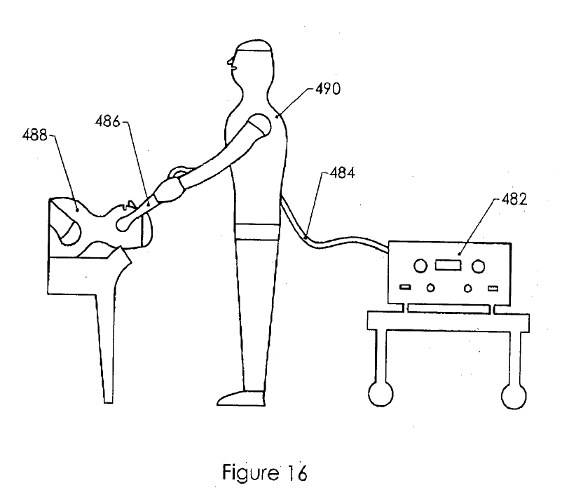

FIG. 16 illustrates a preferred embodiment of the invention system. The System includes an IPL flash light based treatment device 486, electrically fed and in another possible embodiment also water cooled, through a flexible cable 484 by power supply and control unit 482. The 486 IPL based device unit is capable of treating localized acne lesions using an enhanced violet/blue flash light pulsed emission.

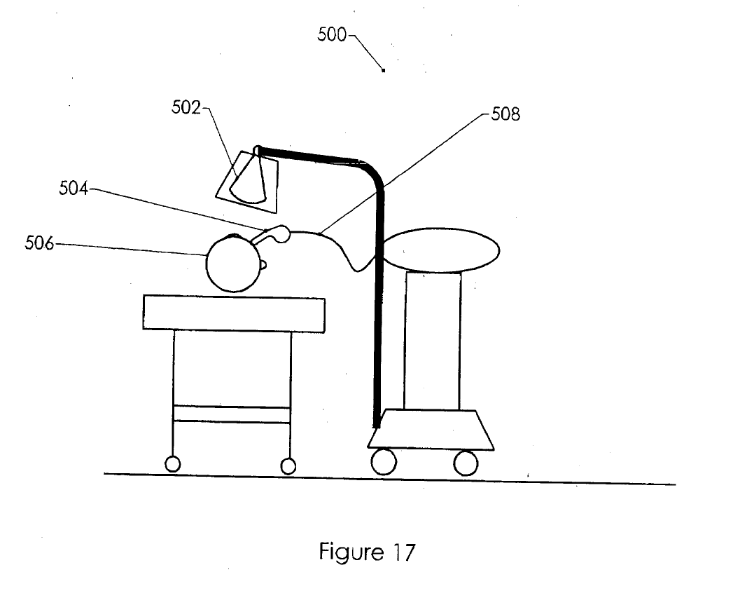

FIG. 17 illustrates another preferred embodiment of the invention system. System 500 includes a high intensity blue/violet light source illumination head 502. Head 502 and is capable of fast eradication of P. acne bacteria by exposing a large skin area of the patient 506 to a high flux of violet/blue light with possible additional spectral bands in the green and red light part of the spectrum, as described in previous embodiments of the invention. The system 500 includes a second IPL flash light treatment device 504, fed through a flexible cable 508. The 506 IPL device unit is capable of treating localized acne lesions using a selectable enhanced violet/blue flash light emission. This 504 device is also capable of reduction the size and appearance of post acne redness in localized areas of the acne affected skin, in the post inflammatory state, using selectable yellow-red flash light emission.

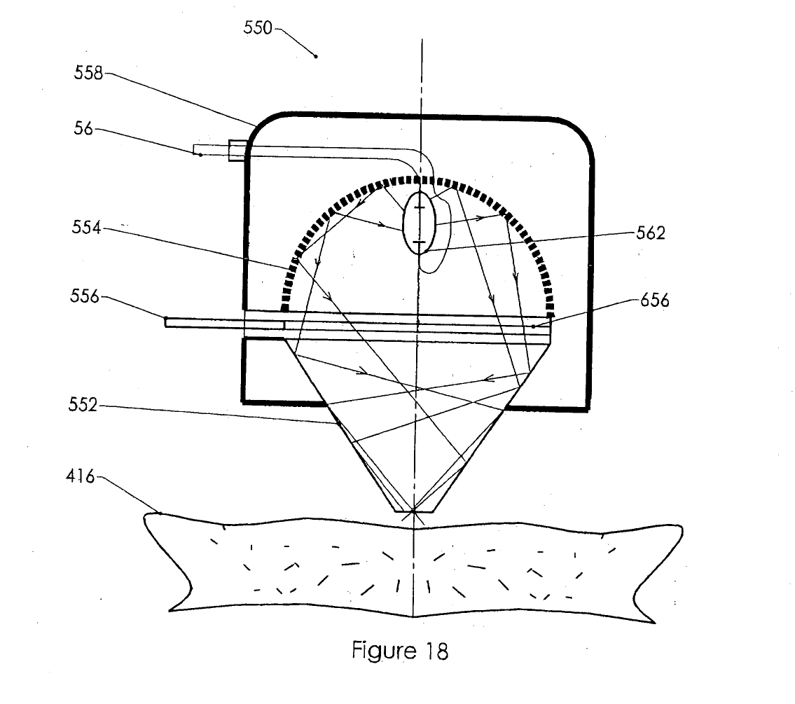

FIG. 18 is another embodiment of the invention high intensity flash lamp based light source 550, is housed in a special packaging case 558, that includes the flash lamp 562 that its arc axis is collinear to the reflector 554 axis, the flash lamp 562 is driven through leads 556. The parabolic reflector 554 has at its aperture a changeable optical filter element 565 equipped with a special rotational lever 556, the filter element 556 has an integrated UV blocking filter on all its spectral selection optional positions and two spectral filtering positions for treatment selection, one for the blue/violet part of the spectrum 400-470 nm and the second for the yellow-red 500-700 nm spectral band. A specially shaped optical light guide 552 collects and directs the selected spectral light beams trough a well-defined cross-section area exit aperture to the treated skin 416.

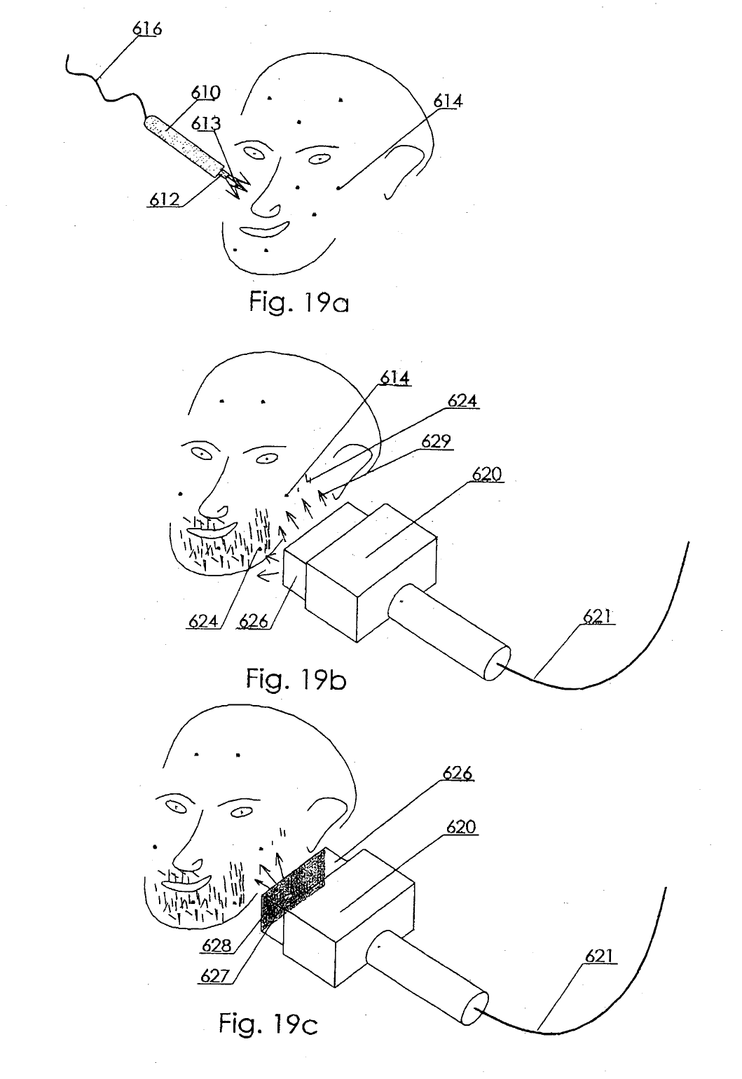

FIG. 19A illustrates an embodiment of the invention where the intense light treatment device for the treatment of post acne inflammation state red spots, is constructed of a relatively small diameter Xenon based flash lamp light source integrated with the ignition H.V. electronic module and a light focusing and beam shaping optics at the treatment end tip of the device 612, a dedicated housing is integrating all these elements into a flash light based hand-piece device 610. The device 610 powered by an external power supply (not shown here) through a feeding flexible cable 616 and is used for the treatment of post acne redness spots 614 on the patient's face skin. The output of the is focused as a concentrated light spot, a spot 613 of typical size in the range of 1-4 mm that does not affect any other area of the face.

In another embodiment of the invention, illustrated in FIG. 19C, a mask 628 with a hole, or an adjustable iris 627, is installed at the distal end of a light guide 626 of an Intense Pulsed Light system such as Quatum™ produced by Lumenis inc. USA, or other similar IPL systems like the one produced by Palomar Inc., USA. The above IPL systems deliver approximately 35 J/cm² and removes sub-cm. vascular lesions by photocoagulation. Typical treatment pulse duration is 2-40 millisecond. In this embodiment we fix a special mask 628 on the treatment aperture side of the IPL head 620 powered through cable 621, while in this mask the typical size of the iris hole 627 is ˜2-4 mm which is the size of most acne lesions 614 and inflamed surroundings. The mask 628 might be made from a thin metal foil like stainless steel, or from a durable plastic cap.

As illustrated in FIG. 19B, a state of the art IPL treatment optical head 620 with an optical lightguide 626, which treats a red lesion 614 but no intently also removes hairs 624 an operation not acceptable for the treatment of males in post acne treatment cases. The addition of the mask 628 shown in the embodiment of FIG. 19C, will therefore spare the facial hairs.

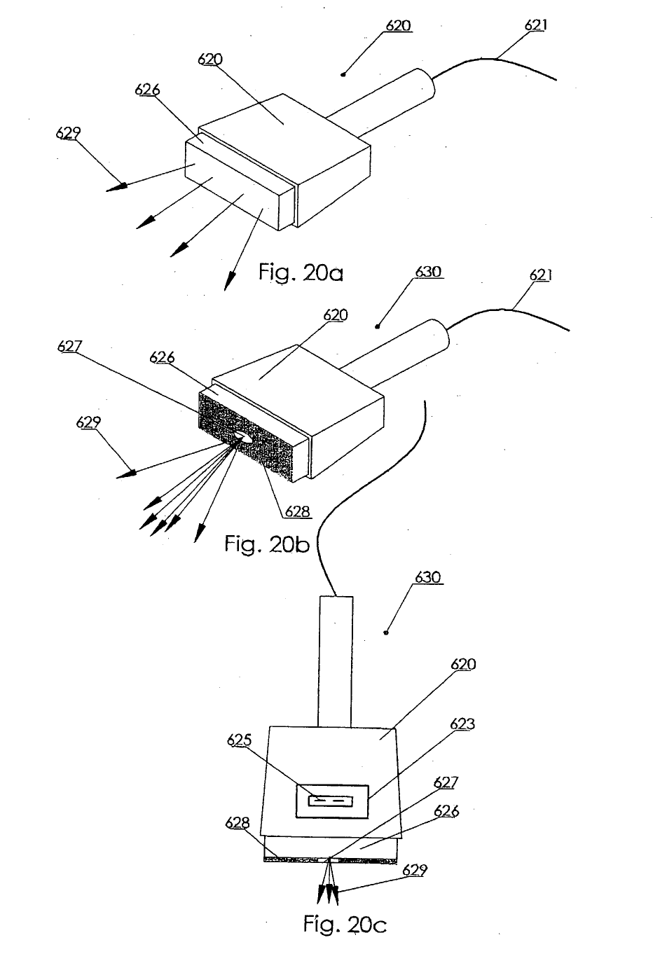

FIGS. 20A-20C illustrate several possible embodiments of the invention related to the use of a rectanular lightguide shape for the treatment of the acne lesion in the two treatment phases namely the P. acne reduction and the redness reduction following phase.

FIG. 20A shows an embodiment of the invention wherein the light pulsed device 620 is including a blue enhanced flash light source within a housing 620 having a rectangular cross-section shape glass lightguide 626 at its exit aperture.

FIG. 20B shows an embodiment of the invention wherein the light pulsed device 630 is including a blue and yellow-red enhanced spectral emittance flash light source fixed within a housing 620 and having a rectangular cross-section shape glass lightguide 626 at its exit aperture. The housing can be fitted with a cap/mask element 628 fixed on the exit aperture of 620, the cap 628 has an iris aperture and has an integrated filter for passing through the iris aperture only the yellow-red light 629 to a defined small exposure area required for reduction of the post acne inflammation skin red spots.

FIG. 20C shows a cross-section view of these embodiment of the invention of device 630 wherein the light pulsed device 630 is including a blue and yellow-red enhanced spectral emittance flash lamp 625 and a reflector 623 fixed within a housing 620 and having a rectangular cross-section shape glass light guide 626 at its exit aperture. The housing is here fitted with a cap/mask element 628 fixed on the exit aperture of 620, the cap 628 has an iris aperture and has an integrated filter for passing through the iris aperture only the yellow-red light 29 to a defined small exposure area required for reduction of the post acne inflammation skin red spots.

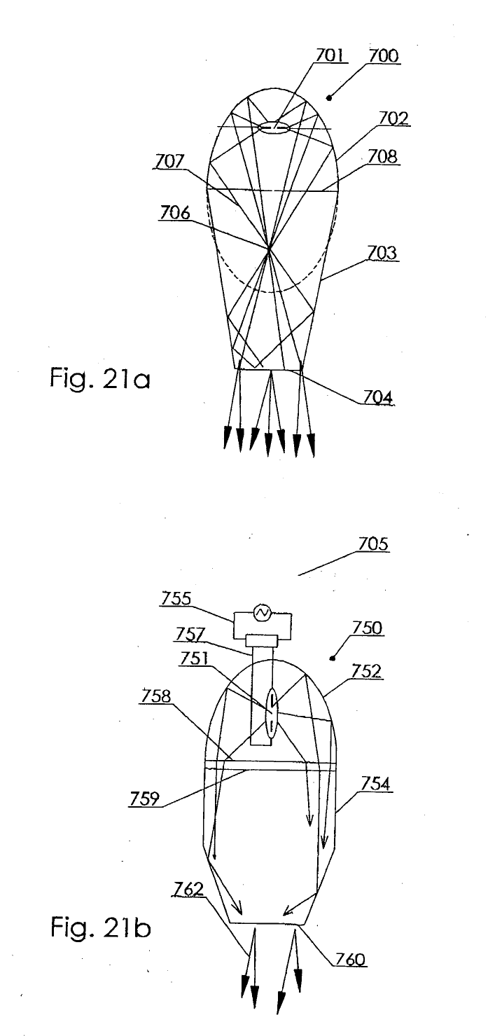

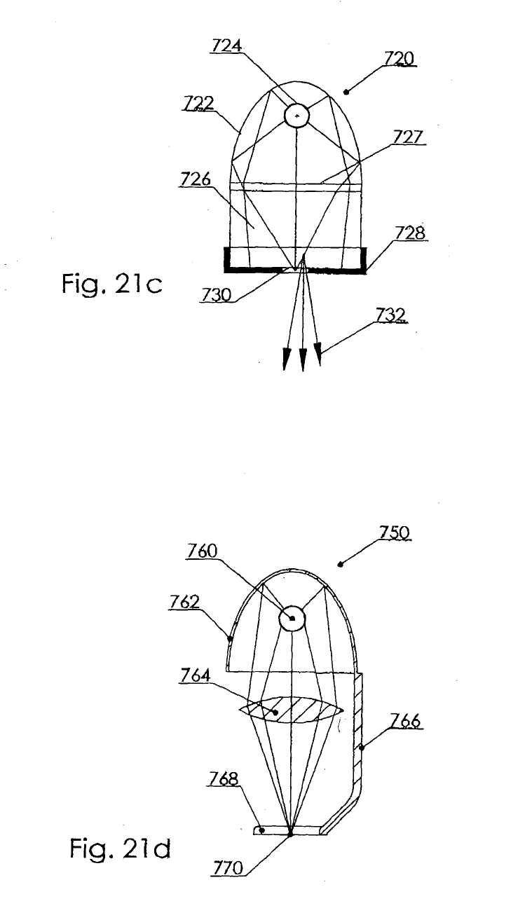

FIG. 21a illustrates several possible embodiments of the invention related to the use a Xenon flash lamp based light source for treating small size vascular lesions typical for post acne red spots.

In FIG. 21A the light source 700 consists of a pulsed miniature xenon flash lamp 702 such as lamp models FX-44XX made by Perkin Elmer. USA, or by Hamamatsu Photonics, Japan; lamps p.n. L7684, L7685, L6605. These lamps have a very short arc length 701 such as 1.5-3 mm length with typical operating pulse voltage of 400-1000 volts. The spectrum of that source is relatively rich with yellow light in the rage 500-550 nm it also have significant spectral content of violet/blue emission in the spectral range 420-450 nm. The light source arc 701 is located and integrated at the focal point of a parabolic, or as in this embodiment an elliptical reflector 702, having a second focal point in 706. The light energy of the flash 707 is transmitted to the lesion with the aid of and through a conical shape glass made light guide 703, which has a relatively small area exit aperture 704. The exit aperture typical cross-section dimension, or effective diameter that might range between 2 mm and 4 mm. The pulse duration of the flash light source is 5 micro seconds which is much shorter when compared to the thermal relaxation time of the 15-50 microseconds of the top skin blood capillaries. The thermal relaxation time of a capillary small diameter blood vessel. The thermal relaxation time of the blood vessel tissue is proportional to the square of the vessel diameter, and is approximately 500 micro-seconds for 50 micron blood vessels diameters. It is therefore 50 microseconds for a 15 micron vessel diameter. As a result, the 5 micro-seconds pulse of energy is confined to the capillary and energy is dissipated to a distance of 300 micron in the surrounding tissue within 40 milliseconds. That energy is absorbed by other blood vessels in the lesion, since the post acne redness is created by a very shallow homogeneous surface distribution of a multitude of capillaries. The energy per pulse of the light source is approximately 100 mj/pulse and the source is operated at a repetition rate of up to 200 pulses per second. The light pulse driving electrical energy is derived from a capacitor bank in the device power supply.

If the flash lamp in the light source is typically operated for about 40 milliseconds the light output energy is accumulated over 8 flash light pulses and creates a total light exposure energy of typically 800 milli-joules for a spot size of 2 mm diameter. Such an exposure procedure creates energy density level of 20 J/cm. Sq known to be sufficient for minor blood vessels coagulation.

FIG. 21B is another embodiment of the invention flash light source 705 wherein the lamp arc 751 axis is collinear to the reflector 752 axis and the lamp 751 is driven through leads 757 by power supply unit module 755. The parabolic reflector 752 has an integrated UV blocking filter 759 at its exit aperture. A specially shaped optical lightguide 754 collects and directs the light beams 762 trough a small cross-section area exit aperture 760.

FIG. 21C is another embodiment of the invention flash light source 720 wherein the lamp 724 is integrated in a parabolic mirror 722 and the light collecting lightguide 726 is of a rectangular shape. The small light aperture required for the redness reduction is created by an add-on cap 728 with an integrated iris element 730 that can be a fixed aperture size or an adaptable size aperture iris.

FIG. 21D is another embodiment of the invention flash light source 780 wherein the lamp 760 is integrated into a curved mirror 762 and the light collecting optical element 764 is lens. The small light aperture required for the redness reduction by high flux light exposure is created by a mechanical fixture 766, 768 that holds the lens at the required place and has an iris aperture 770 at its tip, which is at a distance close to the location of the lens 764 focal point, in order to ensure high energy concentration at the light source exit aperture.

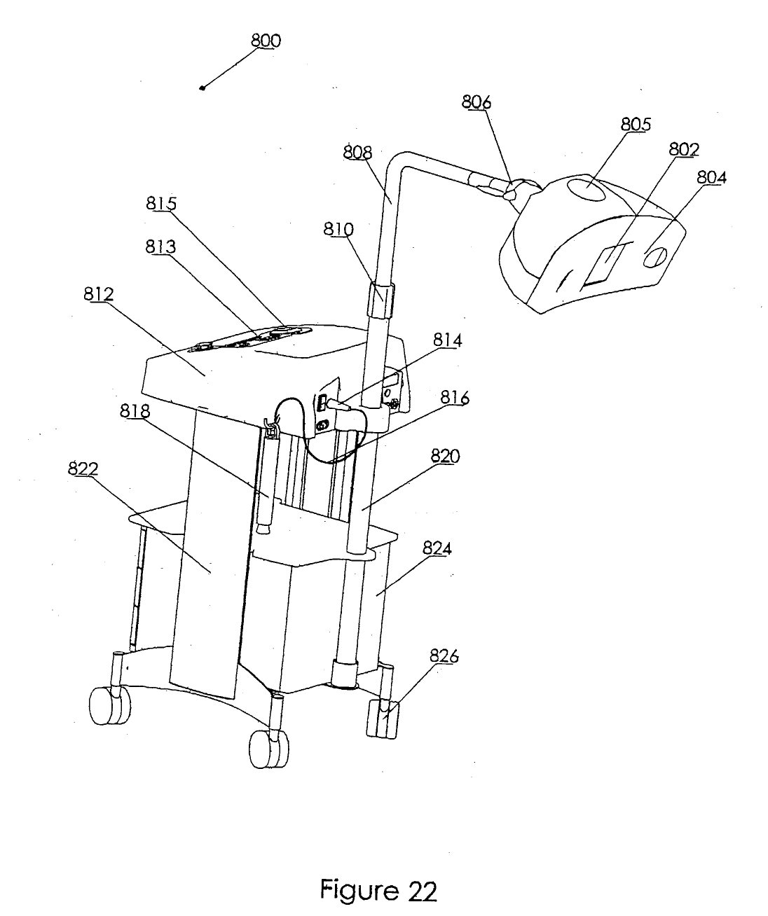

FIG. 22 illustrates another preferred embodiment of the invention system 800 System 800 includes a high intensity blue/violet light source illumination head 804. Head 804 has an active illumination optical aperture 802 and a venting module 805 and is capable of fast eradication of P. acne bacteria by exposing a large skin area of the patient to a high flux of violet/blue light with possible additional spectral bands in the green and red light part of the spectrum, as described in previous embodiments of the invention. The head 804 can be adjusted to the exact level of the treated patient affected skin area by changing the length of pole 808 through level control mechanism adapter 810 and then adapted to the preferred elevation angle by joint 806. The system 800 also includes a small size Xenon flash lamp based light treatment device 818 capable of treating and accelerating the total reduction, or the size and appearance of post acne redness in localized areas of the acne affected skin, in the post inflammatory state. The dual light generating sources 804 and 818 have their associated electronic control and power supply units that are integrated in control module 812 and supply module 824 which are all installed on a wheel 826 based cart module 822 with separate control interfaces 815 and 813 for the P acne eradication blue light source 804 unit and for the IPL redness reduction device 818. The skin redness device 818 is powered through cable 816 and connected to the electronic control unit 812 through the cable 816 connector 814.

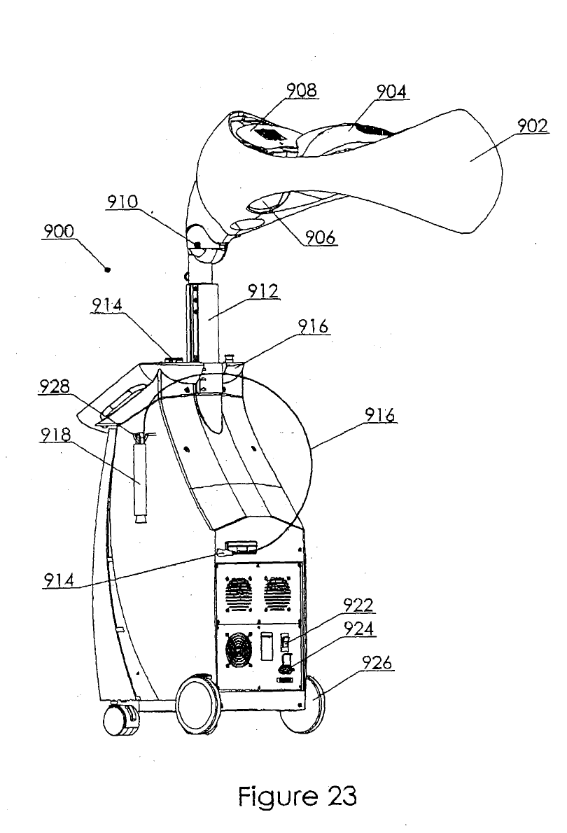

FIG. 23 illustrates another preferred embodiment of the invention system. System 900 includes a high intensity blue/violet dual light source illumination head 902. Head 902 has two active illumination optical illumination apertures 906 (one side only shown here) and two venting modules 908 (one side only shown here) for cooling the head volume. The head 902 is capable of fast eradication of P. acne bacteria by exposing a large skin area of the patient to a high flux of violet/blue light with possible additional spectral bands in the green and red light part of the spectrum, as described in previous embodiments of the invention. The head 902 can be adjusted to the exact level of the treated patient affected skin area by changing the length of pole 912 through a level control electro-mechanism fixed within the chassis module 916. The head 902 can be vertically tilted and adapted to the preferred elevation angle by joint 910. The system 900 also includes a Xenon flash lamp based light treatment device 918 capable of treating and accelerating the total reduction, or the size and appearance of post acne redness in localized areas of the acne affected skin, in the post inflammatory state. The two light generating sources 902 and 918 have their associated electronic control and power supply units that are all integrated in control & supply module 916 which is based on a wheel 926. The system 900 has an integrated computer module with a touch screen that enables flexible control enabling separate computerized control of each of the sources 902 and 918 operational parameters. The P acne eradication is done by the blue light source 804 unit and the redness reduction is done by controlling operational parameters like pulse duration and number of pulses for the IPL based device 918. The skin redness device 918 is powered through cable 916 and connected to the electronic control sub-unit 916 through the cable 916 connector 914.

It should be apparent that there has been provided in accordance with the present invention various inventions that fully satisfy the objectives and advantages set forth above. Although the inventions have been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is to be understood that the invention is not limited in its applications to the details of construction or drawings. The invention is capable of other embodiments, or of being practiced or carried out in various ways. Also, it is to be understood that the phraseology and terminology employed above is for the purpose of description and should not be regarded as limiting. While the invention has been described with respect to a limited number of embodiments, it will be appreciated that many variations, modifications and other applications of the invention may be made.

While the invention has been described with respect to a limited number of embodiments, it will be appreciated that many variations, modifications and other applications of the invention may be made.

What is claimed is:

1. An apparatus for treatment of a skin disorder and conditions and symptoms associated therewith, the apparatus comprising:
(a) at least one light source with spectral emittance concentrated in at least one specific narrow spectral band, wherein one spectral band is in the range of 400 to 450 nm radiating energy fluence of at least 5 J/cm² per a single light exposure; and
(b) an optical system for collecting and shaping light emitted from said at least one light source; and
(c) an electronic unit to control parameters associated with said spectral emittance from said at least one light source.

2. The apparatus of claim 1, wherein an illumination energy of said light source flux, is higher than a predetermined threshold level required for biological destruction of the skin disorder causing factors.

3. The apparatus of claim 1, wherein said light source is an intense pulse light generating source.

4. The apparatus of claim 1, wherein said light source is a time gated high energy CW light source.

5. The apparatus as in claim 1, wherein the skin disorder is caused by p. acne bacteria and said threshold level is a level required for biological destruction of P. acne bacteria at a rate faster than the rate of proliferation.

6. The apparatus of claim 2, wherein said illumination energy threshold level of said illumination light source on a treated skin area is at least 40 mw/cm² for at least 10 minutes and at least 28 Joule/cm² 11.

7. The apparatus of claim 2, wherein said illumination energy threshold level is a level facilitated by a high energy output of a non coherent illumination source; and wherein said illumination source is any combination of radiation sources selected from the group including at least a gas discharge metal halide lamp, a gas discharge xenon lamp, a gas discharge xenon and rare metals combination lamp, an excimer lamp, a LED diode matrix and a diode laser matrix.

8. The apparatus of claim 1, wherein an illuminated area on a patient body by said light source comprises an illumination area large enough to illuminate an infected typical size skin area from a fixed position of said light source related to said skin area.

9. The apparatus of claim 8, wherein the illuminated area on a patient body by said light source is characterized by an energy flux that is above said threshold level and that is large enough in its dimensions to cover and treat in a single exposure session an entire human body part and an entire infected skin area from at least a single fixed position of said light source related to said organ and skin area.

10. The apparatus of claim 1, further comprising:
a computer controlled imaging unit for imaging an illuminated treated area and for monitoring by counting lesions on said treated area, using computerized counting techniques.

11. The apparatus of claim 1, further comprising:
at least one optical element of a group comprising a liquid filled light guide, a solid transparent light guide, a fiber bundle light guide and an array of lenses and mirrors for collecting and conducting the said light source radiation and illuminating the skin treated area at an adjustable distance, energy density and direction.

12. The apparatus of claim 1, wherein said at least one light source is selected from the group including a Gallium, Mercury, xenon and halides gas mixture discharge lamp with peak emission in the 400-450 spectral band, an Excimer lamp with peak emission in the 400-450 spectral band, an Ion Krypton gas laser with a spectral emission in the range 405 to 440 nm, and a diode, wherein said diode is selected from the group consisting of violet/blue laser diodes, and light emitting diodes (LED) with narrow spectral band emission in the range 405-440 nm, or any combination thereof.

13. A method of treating a skin disorder, comprising:
providing a light radiation source having spectral characteristics of at least one of a group of narrow spectral bands consisting of violet/blue (400-450 nm), red (630-670 nm) and green (520-550 nm) light said light source having energy output flux over a predetermined threshold level;
illuminating said skin area with said light radiation source;
additionally illuminating said skin area after a predetermined time period.

14. The method of claim 13, wherein said threshold level is above 5 J/cm².

15. A method according to claim 13, wherein said predetermined time period is at least 24 hours.

16. The method according to claim 13, wherein said skin disorder is selected from the group consisting of acne, seborrhea, soft tissue inflammation, muscular inflammation, post traumatic soft tissue and muscular pain, arthralgia, skin ulcers, contact dermatitis, atopic dermatitis and scleroderma, and conditions and symptoms thereof.

17. The method according to claim 13, wherein said time interval is 1-5 weekly exposures to violet/blue light for typically 2-10 weeks, with a minimum 24 hour's time gap between exposures.

18. The method of claim 13 comprising an intense pulsed light generating source.

19. The apparatus according to claim 4, wherein said treatment comprises treatment of inflammation.

20. The method according to claim 13, wherein treated area is larger than 2 cm×2 cm.

21. A system for the combined treatment of acne inflammatory lesions and for the reduction of the lesion localized redness the system comprising:
(a) a first treatment device for destroying the P. acne bacteria by a violet/blue light exposure; and
(b) a second treatment device for selectively destroying small diameter blood vessels contained at a selected depth and in selected areas of a patient's dermis preferably in the vicinity of the acne lesion.

22. The treatment system of claim 21 wherein at least one of the first and second treatment devices comprises an intense pulse light source.

23. The treatment system of claim 21 wherein the pulsed light source has at least one spectral band between 450 nm to 800 nm, wherein;
(a) each light pulse delivering a fluence at the epidermis surface being treated of between 3 J/cm². and 6 J/cm²;
(b) each pulse having a pulse duration of between 0.2 milliseconds and 2 millisecond;
(c) the exposed skin surface area may be between 0.01 square centimeters and 0.3 square centimeters;
(d) the pulsed light source delivering a single pulse, or a multiple of pulses; and
(e) the light source being selected from the group consisting of a first light source that is continuous-wave light source with pulse duration being controlled by gating the light source to provide a long pulse light and a second light source that is a slow repetition rate narrow pulse source with rate in the range of 0.3 to 2 Hz.
